(19) United States
(12) Patent Application Publication
(10) Pub. No.: US 2025/0066452 A1
(43) Pub. Date: Feb. 27, 2025

(54) SYNTHETIC AMPHIPATHIC HELICAL PEPTIDES AND TREATMENT METHODS USING SYNTHETIC AMPHIPATHIC HELICAL PEPTIDES

(71) Applicants: UNIVERSITY OF MARYLAND, BALTIMORE, Baltimore, MD (US); The United States of America, As Represented by The Secretary, Department of Health and Human Servi, Bethesda, MD (US)

(72) Inventors: Konstantin BIRUKOV, Fulton, MD (US); Thomas L. EGGERMAN, Bethesda, MD (US); Alexander V. BOCHAROV, Bethesda, MD (US); Cynthia L. RENN, Baltimore, MD (US)

(73) Assignees: UNIVERSITY OF MARYLAND, BALTIMORE, Baltimore, MD (US); The United States of America, As Represented by The Secretary, Department of Health and Human Servi, Bethesda, MD (US)

(21) Appl. No.: 18/842,535

(22) PCT Filed: Mar. 2, 2023
(86) PCT No.: PCT/US2023/063601

§ 371 Date: Aug. 29, 2024

Related U.S. Application Data
(60) Provisional application No. 63/316,113, filed on Mar. 3, 2022.

(51) Int. Cl. C07K 14/775; A61K 9/06; A61K 38/00; A61K 45/06; A61P 29/00

(52) U.S. Cl. CPC C07K 14/775; A61K 9/06; A61K 45/06; A61P 29/00; A61K 38/00

(57) ABSTRACT

Therapeutic agents, compositions, and methods are described for use in the treatment of acute or chronic pain, neuroinflammation, or conditions characterized by acute or chronic pain or neuroinflanmmation. The therapeutic agents comprise a synthetic amphipathic helical peptide capable of acting as a mimetic of apoA-I protein.

### CROSS-REFERENCE TO RELATED APPLICATIONS

This application is based on, and claims priority to, U.S. provisional application Ser. No. 63/316,113, filed on Mar. 3, 2022, the entire contents of which is herein incorporated by reference.

### TECHNICAL FIELD

The present disclosure generally relates to synthetic amphipathic helical peptides and treatment methods using synthetic amphipathic helical peptides. For example, the present disclosure provides methods of using the synthetic amphipathic helical peptides to treat acute and chronic pains, including inflammatory pain and neuropathic pain, and to inhibit neuroinflammation.

### BACKGROUND

Chronic and acute pains impose a significant burden on the civilian population, military veterans, and active military personnel in post-wound recovery. Existing opioid and non-opioid pain-managing treatments are only partially effective and cannot be used in a long run due to serious side effects, the most concerning of which are liver chronic toxicity and opioid addiction. Indeed, opioid addiction and abuse are a serious national crisis in the U.S., affecting public health and social and economic welfare. The National Institute on Drug Abuse of the National Institutes of Health estimates that in 2019, nearly 50,000 in the United States died from opioid-involved overdoses (CDC/NCHS, National Vital Statistics System, Mortality. CDC WONDER, Atlanta, GA: US Department of Health and Human Services, CDC; 2019, available at https://wonder.cdc.gov). The opioid crisis makes the task of finding new non-opiates for treating acute and/or chronic pains an urgent priority of paramount importance.

### BRIEF SUMMARY OF INVENTION

The present disclosure provides a method of treating pain in a subject in need thereof, the method comprising administering to the subject a peptide capable of acting as a mimetic of apoA-I protein having the sequence MKAAVLTLAV LFLTGSQARH FWQQDEPPQS PWDRVKDLAT VYVDVLKDSG RDYVSQFEGS ALGKQLNLKL LDNWDSVTST FSKLREQLGP VTQEFWDNLE KETEGLRQEM SKDLEEVKAK VQPYLDDFQK KWQEEMELYR QKVEPLRAEL QEGARQKLHE LQEKLSPLGE EMRDRARAHV DALRTHLAPY SDELRQRLAA RLEALKENGG ARLAEYHAKA TEHLSTLSEK AKPALEDLRQ GLLPVLESFK VSFLSALEEY TKKLNTQ (SEQ ID NO: 31).

In some embodiments, the peptide comprises two sequences selected from the group consisting of DWLKAFYDKVAEKLKEAF (SEQ ID NO: 1), EKLKELLEKLLEKLKELL (SEQ ID NO: 6), ERLLELLRRLLELLRRLL (SEQ ID NO: 27), and a variant or derivative thereof, the two sequences being coupled to each other via a proline or an alanine.

In some embodiments, the peptide comprises a sequence selected from the group consisting of DWLKAFYDKVAEKLKEAFPDWLKAFYDKVAEKLKEAF (SEQ ID NO: 2), DWLKAFYDKVAEKLKEAFPDWAKAAYDKAAEKAKEAA (SEQ ID NO: 3), DHLKAFYDKVACKLKEAFPNWAKAAYDKAAEKAKEAA (SEQ ID NO: 4), DWLKAFYDKVAEKLKEAFPDHAKAAYDKAACKAKEAA (SEQ ID NO: 5), EKLKELLEKLLEKLKELLPEKLKELLEKLLEKLKELL (SEQ ID NO: 7), EKLLELLKKLLELLKKLLPEKLLELLKKLLELLKKLL (SEQ ID NO: 8), EKLKELLEKLLELLKKLLPEKLKELLEKLLELLKKLL (SEQ ID NO: 9), EELKEKLEELKEKLEEKLPEELKEKLEELKEKLEEKL (SEQ ID NO: 10), EELKAKLEELKAKLEEKLPEELKAKLEELKAKLEEKL (SEQ ID NO: 11), EKLKELLEKLKAKLEELLPEKLKELLEKLKAKLEELL (SEQ ID NO: 12), EKLKAKLEELKAKLEELLPEKLKAKLEELKAKLEELL (SEQ ID NO: 13), EKLKALLEKLLAKLKELLPEKLKALLEKLLAKLKELL (SEQ ID NO: 14), EKLKELLEKLLAKLKELLPEKLKELLEKLLAKLKELL (SEQ ID NO: 15), EWLKELLEKLLEKLKELLPEWLKELLEKLLEKLKELL (SEQ ID NO: 16), EKFKELLEKFLEKFKELLPEKFKELLEKFLEKFKELL (SEQ ID NO: 17), EKFKELLEKLLEKLKELLPEKFKELLEKLLEKLKELL (SEQ ID NO: 18), EELKELLKELLKKLEKLLPEELKELLKELLKKLEKLL (SEQ ID NO: 19), EELKKLLEELLKKLKELLPEELKKLLEELLKKLKELL (SEQ ID NO: 20), EKLKELLEKLLEKLKELLAEKLKELLEKLLEKLKELL (SEQ ID NO: 21), EKLKELLEKLLEKLKELLAAEKLKELLEKLLEKLKELL (SEQ ID NO: 22), DWLKAFYDKVACKLKEAFPDWAKAAYNKAAEKAKEAA (SEQ ID NO: 23), DHLKAFYDKVAEKLKEAFPDWAKAAYDKAAEKAKEAA (SEQ TD NO: 24), EKLKAKLEELKAKLEELLPEKAKAALEEAKAKAEELA (SEQ ID NO: 25), EKLKAKLEELKAKLEELLPEHAKAALEEAKCKAEELA (SEQ ID NO: 26), ERLLELLRRLLELLRRLLPERLLELLRRLLELLRRLL (SEQ ID NO: 28), ERLLELLRRLLELLRRLLPDWLKAFYDKVAEKLKEAF (SEQ ID NO: 29), EKLLELLKKLLELLKKLLPDWLKAFYDKVAEKLKEAF (SEQ ID NO: 30), and a variant or derivative thereof. In some embodiments, the peptide comprises DWLKAFYDKVAEKLKEAFPDWLKAFYDKVAEKLKEAF (SEQ ID NO: 2), DWLKAFYDKVAEKLKEAFPDWAKAAYDKAAEKAKEAA (SEQ ID NO: 3), EKLKELLEKLLEKLKELLPEKLKELLEKLLEKLKELL (SEQ ID NO: 7), EKLLELLKKLLELLKKLLPEKLLELLKKLLELLKKLL (SEQ ID NO: 8), EKLKELLEKLLELLKKLLPEKLKELLEKLLELLKKLL (SEQ ID NO: 9), or EKLLELLKKLLELLKKLLPDWLKAFYDKVAEKLKEAF (SEQ ID NO: 30). In some embodiments, the peptide comprises DWLKAFYDKVAEKLKEAFPDWLKAFYDKVAEKLKEAF (SEQ ID NO: 2) or EKLLELLKKLLELLKKLLPDWLKAFYDKVAEKLKEAF (SEQ ID NO: 30).

In some embodiments, the peptide is non-opioid.

In some embodiments, the pain is acute or chronic pain. In some embodiments, the pain is at least one selected from the group consisting of sport-related joint pain, rheumatoid arthritis, burn-related pain, neuropathy, orthopedic pain, phantom pain in amputees and foot neuro-degenerative syndrome.

In some embodiments, the peptide is administered topically, intravenously, or intramuscularly. In some embodiments, the peptide is administered topically as a lotion or a hydrogel. In some embodiments, an amount of the peptide in the lotion or the hydrogel is 100 μg/ml to 1000 μg/ml, preferably 100 μg/ml. In some embodiments, the daily dosage by topical administration is 0.5 mg/kg to 10 mg/kg, preferably about 1-2 mg/kg body weight. In some embodiments, an amount of the peptide in the intramuscular injection is 0.001 mg/ml to 100 mg/ml, preferably 3 mg/ml. In some embodiments, the daily dosage by intramuscular injection is 0.5 mg/kg to 100 mg/kg, preferably about 10 mg/kg. In some embodiments, an amount of the peptide in the intravenous administration is 0.001 mg/ml to 100 mg/ml, preferably 3 mg/ml. In some embodiments, the daily dosage by intravenous administration is 0.5 mg/kg to 100 mg/kg, preferably about 10-50 mg/kg. In some embodiments, the peptide is administered once daily.

In some embodiments, the peptide is comprised in a pharmaceutical composition, the pharmaceutical composition further comprising a pharmaceutically acceptable carrier.

In some embodiments, the pharmaceutical composition further comprises at least one of a non-steroidal anti-inflammatory drug and an opioid analgesic.

The present disclosure also provides a method of treating neuroinflammation in a subject in need thereof, the method comprising administering to the subject a peptide capable of acting as a mimetic of apoA-I protein having the sequence MKAAVLTLAV LFLTGSQARH FWQQDEPPQS PWDRVKDLAT VYVDVLKDSG RDYVSQFEGS ALGKQLNLKL LDNWDSVTST FSKLREQLGP VTQEFWDNLE KETEGLRQEM SKDLEEVKAK VQPYLDDFQK KWQEEMELYR QKVEPLRAEL QEGARQKLHE LQEKLSPLGE EMRDRARAHV DALRTHLAPY SDELRQRLAA RLEALKENGG ARLAEYHAKA TEHLSTLSEK AKPALEDLRQ GLLPVLESFK VSFLSALEEY TKKLNTQ (SEQ ID NO: 31).

In some embodiments, the peptide comprises two sequences selected from the group consisting of DWLKAFYDKVAEKLKEAF (SEQ ID NO: 1), EKLKELLEKLLEKLKELL (SEQ ID NO: 6), ERLLELLRRLLELLRRLL (SEQ ID NO: 27), and a variant or derivative thereof, the two sequences being coupled to each other via a proline or an alanine.

In some embodiments, the peptide consists of a sequence selected from the group consisting of DWLKAFYDKVAEKLKEAFPDWLKAFYDKVAEKLKEAF (SEQ ID NO: 2), DWLKAFYDKVAEKLKEAFPDWAKAAYDKAAEKAKEAA (SEQ ID NO: 3), DHLKAFYDKVACKLKEAFPNWAKAAYDKAAEKAKEAA (SEQ ID NO: 4), DWLKAFYDKVAEKLKEAFPDHAKAAYDKAACKAKEAA (SEQ ID NO: 5), EKLKELLEKLLEKLKELLPEKLKELLEKLLEKLKELL (SEQ ID NO: 7), EKLLELLKKLLELLKKLLPEKLLELLKKLLELLKKLL (SEQ ID NO: 8), EKLKELLEKLLELLKKLLPEKLKELLEKLLELLKKLL (SEQ ID NO: 9), EELKEKLEELKEKLEEKLPEELKEKLEELKEKLEEKL (SEQ ID NO: 10), EELKAKLEELKAKLEEKLPEELKAKLEELKAKLEEKL (SEQ ID NO: 11), EKLKELLEKLKAKLEELLPEKLKELLEKLKAKLEELL (SEQ ID NO: 12), EKLKAKLEELKAKLEELLPEKLKAKLEELKAKLEELL (SEQ ID NO: 13), EKLKALLEKLLAKLKELLPEKLKALLEKLLAKLKELL (SEQ ID NO: 14), EKLKELLEKLLAKLKELLPEKLKELLEKLLAKLKELL (SEQ ID NO: 15), EWLKELLEKLLEKLKELLPEWLKELLEKLLEKLKELL (SEQ ID NO: 16), EKFKELLEKFLEKFKELLPEKFKELLEKFLEKFKELL (SEQ ID NO: 17), EKFKELLEKLLEKLKELLPEKFKELLEKLLEKLKELL (SEQ ID NO: 18), EELKELLKELLKKLEKLLPEELKELLKELLKKLEKLL (SEQ ID NO: 19), EELKKLLEELLKKLKELLPEELKKLLEELLKKLKELL (SEQ ID NO: 20), EKLKELLEKLLEKLKELLAEKLKELLEKLLEKLKELL (SEQ ID NO: 21), EKLKELLEKLLEKLKELLAAEKLKELLEKLLEKLKELL (SEQ ID NO: 22), DWLKAFYDKVACKLKEAFPDWAKAAYNKAAEKAKEAA (SEQ ID NO: 23), DHLKAFYDKVAEKLKEAFPDWAKAAYDKAAEKAKEAA (SEQ ID NO: 24), EKLKAKLEELKAKLEELLPEKAKAALEEAKAKAEELA (SEQ ID NO: 25), EKLKAKLEELKAKLEELLPEHAKAALEEAKCKAEELA (SEQ ID NO: 26), ERLLELLRRLLELLRRLLPERLLELLRRLLELLRRLL (SEQ ID NO: 28), ERLLELLRRLLELLRRLLPDWLKAFYDKVAEKLKEAF (SEQ ID NO: 29), EKLLELLKKLLELLKKLLPDWLKAFYDKVAEKLKEAF (SEQ ID NO: 30), and a variant or derivative thereof. In some embodiments, the peptide comprises DWLKAFYDKVAEKLKEAFPDWLKAFYDKVAEKLKEAF (SEQ ID NO: 2), DWLKAFYDKVAEKLKEAFPDWAKAAYDKAAEKAKEAA (SEQ ID NO: 3), EKLKELLEKLLEKLKELLPEKLKELLEKLLEKLKELL (SEQ ID NO: 7), EKLLELLKKLLELLKKLLPEKLLELLKKLLELLKKLL (SEQ ID NO: 8), EKLKELLEKLLELLKKLLPEKLKELLEKLLELLKKLL (SEQ ID NO: 9), or EKLLELLKKLLELLKKLLPDWLKAFYDKVAEKLKEAF (SEQ ID NO: 30). In some embodiments, the peptide comprises DWLKAFYDKVAEKLLKEAFPDWLKAFYDKVAEKLKEAF (SEQ ID NO: 2) or EKLLELLKKLLELLKKLLPDWLKAFYDKVAEKLKEAF (SEQ ID NO: 30).

In some embodiments, the peptide is administered topically, intravenously, or intramuscularly.

### BRIEF DESCRIPTIONS OF DRAWINGS

The subject matter that is regarded as the invention is particularly pointed out and distinctly claimed in the claims at the conclusion of the specification. The objects, features, and advantages of the present disclosure are apparent from the following detailed description taken in conjunction with the accompanying drawings in which:

FIG. 1 shows comparative structural analyses of embodiments of the synthetic amphipathic helical peptides (SAHP) of the present disclosure. The top left of FIG. 1 shows the structure of the L37pA peptide having the sequence DWLKAFYDKVAEKLKEAFPDWLKAFYDKVAEKLKEAF (SEQ ID NO: 2). The top right of FIG. 1 shows the structure of the 5A peptide having the sequence DWLKAFYDKVAEKLKEAFPDWAKAAYDKAAEKAKEAA (SEQ ID NO: 3). The bottom left of FIG. 1 shows the structure of the ELK peptide having the sequence EKLKELLEKLLEKLKELLPEKLKELLEKLLEKLKELL (SEQ ID NO: 7). The bottom right of FIG. 1 shows the structure of the ELK-B peptide having the sequence EKLLELLKKLLELLKKLLPEKLLELLKKLLELLKKLL (SEQ ID NO: 8). Hydrophobic residues are shown in light gray, and hydrophilic residues are shown in dark gray.

FIGS. 2(a) to 2(c) show the chronic analgesic effects of an intramuscular injection of the synthetic amphipathic helical peptide L37pA in Complete Freund's Adjuvant (CFA) animal model of chronic inflammatory pain. L37pA, inactive peptide L3D-L37pA, and vehicle were administered as an intramuscular injection every consecutive day 30 minutes prior to behavioral test of CFA-injected or saline-injected plantar surface of left hind paw mice. Injections of CFA and peptides were performed in left paws. Right paws of the animals remained intact and were used for comparison. Pain was assessed as a decreased paw withdrawal temperature. FIG. 2(a) shows that intramuscular injection of peptide L37pA reduces inflammatory heat hyperalgesia. FIG. 2(b) shows the thickness of CFA-injected left paws in all four test groups of mice and FIG. 2(c) shows that the thickness of untreated right paws in all four groups of mice is similar at all the time points. FIGS. 2(b) and 2(c) show that the pain-reducing effects of the L37pA peptide are independent of its anti-inflammatory properties. The thicknesses of the left and right hind paws of the mice injected with CFA were monitored. Data showed that the pain-reducing effects of the L37pA peptide could be detected as early as 30 minutes post-administration and did not affect the limb inflammatory swelling caused by CFA.

FIGS. 3(a) to 3(c) show the chronic analgesic effects of a topical application of the synthetic amphipathic helical peptide L37pA in an animal model of chronic inflammatory pain caused by local injection of Complete Freund's Adjuvant (CFA). Five different test groups of mice were administered saline intramuscular injection and topical application of L3D-L37pA, vehicle (saline) intramuscular injection and topical application of L37pA, CFA paw injection and topical application of vehicle, CFA paw injection and topical application of L3D-L37pA, and CFA paw injection and topical application of L37pA. The topical application of the peptides was carried out 3 hours after the vehicle or CFA injection and applied once a day in the following days. The peptides were applied 30 minutes before thermosensitivity measurement. FIG. 3(a) shows that the topical application of L37pA reduces heat hyperalgesia in the CFA model of inflammatory pain. FIG. 3(b) shows that the topical application of L37pA does not affect CFA-induced increase in left paw thickness, and FIG. 3(c) shows that in the five test groups of mice, the untreated right paws had the similar thickness at all the time points. FIGS. 3(b) and 3(c) show that the pain-reducing effects of the L37pA peptide are independent of its anti-inflammatory properties. The thicknesses of the left and right hind paws of the mice injected with CFA were monitored. Data showed that the pain-reducing effects of the L37pA peptide could be detected as early as 30 minutes post-administration and did not affect the limb inflammatory swelling caused by CFA.

FIG. 4 shows the comparative chronic analgesic effects of an intramuscular injection and a topical application of the synthetic amphipathic helical peptide L37pA in an animal model of chronic inflammatory pain caused by Complete Freund's Adjuvant (CFA). Six different test groups of mice were administered a vehicle (saline) injection and an intramuscular injection of L3D-L37pA, a vehicle (saline) injection and an intramuscular injection of L37pA, a CFA injection and an intramuscular injection of L3D-L37pA, a CFA injection and an intramuscular injection of L37pA, a CFA injection and a topical application of vehicle, and a CFA injection and a topical application of L37pA. The topical application of the peptides was carried out 3 hours after the vehicle (saline) or CFA injection and applied once a day in the following days. The peptides were applied 30 minutes before thermosensitivity assessment.

FIGS. 5(a) to 5(e) show the chronic analgesic effects of topical applications of the five different synthetic amphipathic helical peptide in an animal model of chronic inflammatory pain caused by Complete Freund's Adjuvant (CFA). In each of the figures, four different test groups of mice were administered a saline injection and a topical application of the peptide of interest, a vehicle (saline) injection and a topical application of L3D-L37pA, a CFA injection and a topical application of the peptide of interest, and a CFA injection and a topical application of L3D-L37pA. The topical application of the peptides was carried out 3 hours after the vehicle or paw CFA injection and applied once a day in the following days. More specifically, the peptides were first applied 3 hours after the CFA injection, and measurement of thermosensitivity was performed 30 minutes post-peptide treatment. Afterwards, the peptides were applied once a day. FIG. 5(a) shows experiments with the synthetic peptide L37pA. FIG. 5(b) shows experiments with the synthetic peptide ELK-B-P-18A. FIG. 5(c) shows experiments with the synthetic peptide ELR-P-18A. FIG. 5(d) shows experiments with the synthetic peptide ELR-B. FIG. 5(e) shows experiments with the synthetic peptide ELK-B.

FIGS. 6(a) and 6(b) show the chronic analgesic effects of a topical application of the synthetic amphipathic helical peptide L37pA in animal model of chronic neuropathic pain. Mice were anesthetized and a longitudinal incision was made in the left hips of the mice to expose the proximal sciatic nerve. In the sham mouse group, the skin incision was closed by sterile suture and Vetbond glue. In the PSNL mouse group, the sterile suture was passed through the dorsal 1/3 of the sciatic nerve and then tied for litigation. Mechanical allodynia test was performed before surgery and at 7 days post operation. A topical formulation containing 0.2 mg/ml of the L37pA peptide was prepared. A negative control was preparing using a vehicle without any peptide. The topical formulation and the negative control were administered to the respective mice at day 8 post-operation. The mechanical allodynia test was performed before the topical application and at 30 minutes after application at days 3, 7, and 14 after starting the topical treatment. The data show statistically significant reversal of pain sensation in response to stimuli by the topical application of the L37pA peptide. The administration of the L37pA peptide is able to produce sustained pain-relieving effects. FIG. 6(a) shows that repetitive L37pA daily topical skin application reduces paw withdrawal threshold in PSNL model. FIG. 6(b) shows that repetitive L37pA daily skin application in PSNL model—control tests.

FIGS. 7(a) and 7(b) show that the L37pA peptide alleviates neuropathic pain induced mechanical allodynia. Partial sciatic nerve ligation (PSNL) on left side was performed as neuropathic pain model in this study. Mechanical allodynia was evaluated prior study (baseline, BL), at 7d post operation (7d post opt), pre and post 30 minutes peptide treatment at 3d, 7d, and 14d. Vehicle or 0.2 mg/ml L37pA peptide was topically applied on the left hind paw every consecutive day. All mice were applied vehicle on the right hind paw every consecutive day as sensitization control. FIG. 7(a) shows mechanical allodynia from the left hind paw. FIG. 7(b) shows mechanical allodynia from the right hind paw. Results from mechanical allodynia are presented as withdrawal threshold (in g) mean: SEM, n=4 per group (*p<0.05 vs. sham/veh; * p<0.01 vs. sham/veh).

FIG. 8 shows that lipopolysaccharide (LPS) and various damage-associated molecular patterns (DAMPs) induce a dose-dependent pro-inflammatory response that is CD36-dependent. Following 20 hours incubation of wild-type (WT) and CD36-expressing (CD36+) HEK293 cells with increasing concentrations of CD36 ligands, the IL-8 levels were determined in conditioned medium by ELISA.

FIGS. 9(a)-9(c) show that the SAMP L37pA can inhibit the CD36-dependent PAMP/DAMP pro-inflammatory response. Following 20 hours incubation of CD36-expressing cells with the indicated amounts of the ligands, the IL-8 levels were determined in conditioned medium. The cells were incubated with (i) the indicated amount of the ligand alone, (ii) the indicated amount of the ligand in combination with the active SAMP L37pA, and (iii) the indicated amount of the ligand with the inactive SAMP L3D. FIG. 9(a) shows the PAMP lipopolysaccharide (LPS), FIG. 9(b) shows the DAMP high mobility group box protein 1 (HMGB1), and FIG. 9(c) shows the DAMP Histone 3B.

### DETAILED DESCRIPTION

The pathologic mechanisms of acute pain are not fully elucidated. Even less is known about the origins of chronic pain. It has been proposed that pain sensation is triggered by pain receptors at peripheral nerve endings at the distal end of an axon, and that signs of chronic inflammation in the nerve endings may be associated with developing sustained pain.

The applicant hypothesizes that sub-chronic damage of neurons and surrounding tissue under such inflammatory conditions leads to local release of damage-associated molecular patterns (DAMPs), and this local release of DAMPs perpetuates a vicious cycle of inflammation leading to increased release of DAMPs, pain-inducing chemokines, escalating pain sensation (excitation of pain neuronal activity), and/or hypersensitivity of pain receptors. DAMPs are a diverse group of biomolecules that originate from various cellular compartments. These biomolecules typically have a different specific function during normal cellular activity, but when the cell detects a stress stimulus, they are activated now functioning to signal “the alarm”. Many different molecules are classified as DAMPs, including thromboplastin, acute phase proteins, serum amyloid A (SAA), heat shock proteins including HSP60, HSP70, and glucose-regulated protein 94 (GRP94), and high mobility group box protein 1 (HMGB1). DAMPs initiate inflammation through their interaction with innate immune response receptors such as toll-like receptors (TLRs) (Kato J. and Svesson, C. I., Prog. Mol. Biol. Transl. Sci., 13:251-279 (2015)). TLRs not only interact with DAMPs, but they also detect pathogen-associated molecular patterns (PAMPs) during bacterial and viral infections.

A release of DAMPs such as thromboplastin, acute phase proteins, SAA, heat shock proteins including HSP60, HSP70, GRP94, and HMGB1 has been observed in septic and aseptic models of inflammation, trauma or crush syndrome. Elevation of HMGB1 and other DAMP compounds and molecules has been documented in patients with sepsis or in trauma victims. Blockade of extracellular HMGB1 activity by small molecule compounds has been observed to reduce pain in several animal models (Kato J. and Svesson, C. I., Prog. Mol. Biol. Transl. Sci., 13:251-279 (2015)).

Apolipoproteins are lipoprotein (LP)-associated proteins that stabilize LP structure and mediate receptor-dependent LP recognition. LP's interaction with receptors, such as the low-density LP (LDL receptor or class B scavenger receptors (SR-Bs; SR-BI, SR-BII (the splicing variant of SR-BI), or CD36) controls many aspects of lipid metabolism. In addition, SR-Bs mediate pathogen recognition and innate and adaptive immune response (Bocharov, A. V., et al., J. Immunol., 197:611-619 (2016); Bocharov, A. V., et al., J. Biol. Chem., 279:36072-36082 (2004)). This class of SR-B which include SR-BI, SR-BII and CD36 have been demonstrated to participate in an innate immune response by recognizing and mediating downstream signaling and clearance of a number of DAMPs and PAMPs.

Apolipoprotein A-I (apoA-I) is the major protein of high-density LP (HDL), which plays an important role in reverse cholesterol transport, as well as possesses anti-inflammatory and tissue-protecting properties. apoA-I has been known to attenuate atherosclerosis via reverse cholesterol transport from macrophages residing in atherosclerotic plaques, as well as reducing inflammation triggered by reactive oxygen species, oxidized LPs, various proinflammatory bacteria-derived products, bacteria, and acute-phase proteins. apoA-I exerts these effects in part by blocking various receptors sensing DAMPs (Bocharov, A. V., et al., J. Immunol., 197:611-619 (2016); Bocharov, A. V., et al., J. Biol. Chem., 279:36072-36082 (2004); Remaley, A. T., et al., J. Lipid Res., 44:828-836 (2003)). while the therapeutic benefits of apoA-I are recognized, apoA-I is impractical for use as a therapeutic agent because pharmaceutical-grade apoA-I can be prohibitively expensive (Bocharov, A. V., et al., J. Immunol., 197:611-619 (2016)).

Synthetic amphipathic helical peptides (SAHPs) are apoA-I mimetic peptides because of their ability to mediate some of apoA-I protein's functions. More particularly, these SAHPs have been initially developed to mimic apoA-I in activating lecithin:cholesterol acyltransferase (LCAT), facilitating cholesterol efflux and reverse cholesterol transport resulting in an increased cholesteryl ester transport to the liver, a reduction of circulating LDL, VLDL and oxLDL. To specifically function as apoA-I mimetic peptide, the SAHPs have an amphipathic α-helical structure that is similar to the native apoA-I protein secondary structure containing 10 such amphipathic α helices. Certain SAHPs have been shown to antagonize various apoA-I binding receptors, such as formyl peptide receptors, lectin-like oxidized low-density LP receptor (LOX-1), and class A and class B scavenger receptors including SR-BI, SR-BII, and CD36 (Bocharov, A. V., et al., J. Immunol., 197:611-619 (2016)). Preclinical studies showed SAHP effects in reducing atherosclerotic lesion formation, attenuation of atherosclerotic vascular inflammation in mice.

The applicant has discovered and developed novel SAHPs that selectively target CD36 as well as SR-BUII to inhibit CD36 and/or SR-BUII inflammatory signaling, and in particular, the applicant previously tested a panel of SAHPs that effectively and remarkably reduced LPS-induced inflammation and endothelial barrier dysfunction in vitro and in vivo (Bocharov, A. V., et al., J. Immunol., 197:611-619 (2016)). The applicant has identified certain of the novel SAHPs that significantly reduced the magnitude of LPS-induced acute lung injury in mouse models (Bocharov, A. V., et al., J. Immunol., 197:611-619 (2016)). The applicant has shown that the anti-inflammatory effects of the novel SAHPs are facilitated by SAHP binding to CD36 and CD36-associated endocytosis of bacterial pathogens and suppression of inflammatory pathway (Bocharov, A. V., et al., J. Immunol., 197:611-619 (2016)).

Based on the applicant's hypothesis that sub-chronic damage of neurons and surrounding tissue under such inflammatory conditions leads to local release of DAMPs such as HMGB1, SAA, and heat shock protein HSP60, the applicant was surprised to find that treatment with the novel SAHPs makes it possible to not only markedly increase threshold of pain response in animal models of acute and chronic pains, including animal models of inflammatory and neuropathic pains, but to also inhibit neuroinflammation.

The synthetic amphipathic helical peptides and treatment methods using the synthetic amphipathic helical peptides according to embodiments within the present application are described in further detail below.

### Definitions

The terminologies used in the present application are for purpose of describing the particular embodiments and are not intended to be limiting. Unless defined otherwise or clearly specified otherwise by their use in the present application, all technical and scientific terms used in this application have meanings commonly used in the art. As used in this application, the following words or phrases having the meanings specified.

As used in this application, “apoA-I” refers to full-length and unmodified apoA-I, unless context clearly indicates otherwise. For example, “apoA-I peptides” refer to small portions of full-length apoA-I. Typically, the apoA-I is a human apoA-I, a 28.2 kDa protein of 244 amino acids, as shown in Table 1:

TABLE 1
Human apoA-I amino acid sequence
[NCBI RefSeq accession number
NP_000030.1] (SEQ ID NO: 31)

```
    1 MKAAVLTLAV LFLTGSQARH FWQQDEPPQS
        PWDRVKDLAT VYVDVLKDSG RDYVSQFEGS
  61 ALGKQLNLKL LDNWDSVTST FSKLREQLGP
        VTQEFWDNLE KETEGLRQEM SKDLEEVKAK
121 VQPYLDDFQK KWQEEMELYR QKVEPLRAEL
        QEGARQKLHE LQEKLSPLGE EMRDRARAHV
181 DALRTHLAPY SDELRQRLAA RLEALKENGG
        ARLAEYHAKA TEHLSTLSEK AKPALEDLRQ
241 GLLPVLESFK VSFLSALEEY TKKLNTQ
```

As used in this application, “apoA-I mimetic” and “apoA-I mimetic peptide” are used interchangeably to refer to apolipoprotein A-I mimicking peptide. apoA-I mimetics are peptides that are functionally and/or structurally mimic apoA-I.

As used in this application, “18A” refers to the peptide DWLKAFYDKVAEKLKEAF (SEQ ID NO: 1), when used in the context of a peptide or peptide sequence. The peptide sequence can occur as an isolated peptide, or as a sequence within a larger peptide sequence.

As used in this application, “amphipathic helical peptide” refers to a peptide comprising at least one amphipathic helix (amphipathic helical domain). In some embodiments, the amphipathic helical peptides of the present disclosure comprise two or more amphipathic helices.

As used in this application, “class A amphipathic helix”, “type A amphipathic α helix”, and “amphipathic α helix” are used interchangeably to refer to a protein structure that forms an α-helix producing a segregation of hydrophobic and hydrophilic on opposite surfaces of the α-helix.

As used in this application, “monomer”, “monomeric”, and “monomeric form”, when used in the context of a peptide or peptide sequence, are used interchangeably to refer to an amphipathic helical peptide having one type A amphipathic α helix.

As used in this application, “dimer”, “dimeric”, and “dimeric form”, when used in the context of a peptide or peptide sequence, are used interchangeably to refer to an amphipathic helical peptide having two type A amphipathic α helices joined by a linker group.

As used in this application, “polypeptide”, “peptide”, and “protein” are used interchangeably to refer to a polymer of amino acid residues, whether isolated from natural sources, produced by recombinant techniques, or chemically synthesized.

As used in this application, “variant” in the context of a peptide or peptide sequence refers either to a naturally occurring allelic variation of a given peptide or a chemically synthesized variation of a given peptide or protein in which one or more (e.g. one, two, or three) amino acid residues have been modified by amino acid substitution, addition, or deletion. The variants are preferably capable of treating acute or chronic pain, neuroinflammation, or conditions characterized by acute or chronic pain and/or neuroinflammation, or are active in experimental models of acute or chronic pain, neuroinflammation, and/or conditions characterized by those symptoms, for example, by acting as inhibitors of the activity of receptors such as HMGB1, HSP60, and SAA.

As used in this application, “derivative” in the context of a peptide or peptide sequence refers to a variation of given peptide or protein that are otherwise modified, i.e., by covalent attachment of any type of molecule, preferably having bioactivity, to the peptide or protein, including non-naturally occurring amino acids. The derivatives are preferably capable of treating acute or chronic pain, neuroinflammation, or conditions characterized by acute or chronic pain and/or neuroinflammation, or are active in experimental models of acute or chronic pain, neuroinflammation, and/or conditions characterized by those symptoms, for example, by acting as inhibitors of the activity of receptors such as HMGB1, HSP60, and SAA.

As used in this application, “a” or “an” means at least one, unless clearly specified otherwise.

### Synthetic Amphipathic Helical Peptides

The present disclosure provides novel synthetic amphipathic helical peptides (SAHPs) that are apoA-I mimetic peptides. The peptides have both hydrophobic and hydrophilic domains, and an amphipathic helix in the structure. In designing the peptides, the applicant has found that minimal changes in the apoA-J mimetic peptide sequence affecting polar and nonpolar interfaces oriented along the long axis of the peptide helix region may change specific interactions with various receptors. For example, peptides of the present disclosures are designed based on combinations of features such as net charge, mean hydrophobicity, hydrophobic phase size, type of helix, and/or configuration of the linker bridge between the helices. Peptides of the present disclosure can be broadly categories into three families: first, 18A-based peptides; second, ELK-based peptides; and third, ELR-based peptides.

### 18A-Based Peptides

In some embodiments, the peptide may comprise the monomeric form of the 18A peptide having the amino acid sequence DWLKAFYDKVAEKLKEAF (SEQ ID NO: 1). The 18A peptide forms a type A amphipathic helix. The 18A-based peptides including L37pA (SEQ ID NO: 2) has been shown to exhibit anti-pain and anti-swelling effects in the CFA model of inflammatory pain.

In some embodiments, the peptide may be a homodimeric peptide having two identical copies of the 18A peptide coupled through a linker. The linker may be proline. The peptide may be a heterodimeric peptide having the 18A peptide and a modified 18A peptide coupled through a linker. The linker may be proline. The SAHP may be heterodimeric peptide having two modified 18A peptides coupled through a linker. The amino acid sequences of the two modified 18A peptides may be the same or different. The linker may be proline. In some embodiments, the peptide consists of L-amino acids.

In some embodiments, the peptide has the amino acid sequence shown in Table 2:

TABLE 2

| Peptide | | SEQ ID |
|---|---|---|
| Name | Amino Acid Sequence | NO: |
| 18A | DWLKAFYDKVAEKLKEAF | 1 |
| L37pA | DWLKAFYDKVAEKLKEAF | 2 |
| | PDWLKAFYDKVAEKLKEA | |
| | F | |
| 5A | DWLKAFYDKVAEKLKEAF | 3 |
| | PDWAKAAYDKAAEKAKEA | |
| | A | |
| P5A | DHLKAFYDKVACKLKEAF | 4 |
| | PNWAKAAYDKAAEKAKEA | |
| | A | |
| P5A | DWLKAFYDKVAEKLKEAF | 5 |
| C12/H2 | PDHAKAAYDKAACKAKEA | |
| | A | |

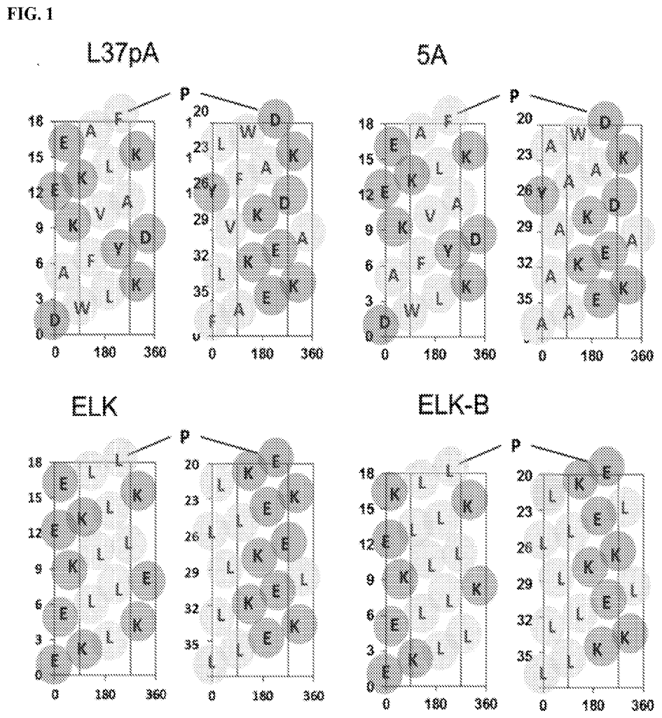

FIG. 1 shows the comparative structural analyses of the L37pA and 5A peptides. The L37pA is a homodimeric peptide having two identical copies of the 18A peptides linked by proline. The peptide 5A is a heterodimeric peptide having two type A amphipathic α helices (18A and modified 18A) linked by a proline. Five amino acids in 18A are substituted with alanine to modulate the hydrophobicity of the helix corresponding to the modified 18A. Peptides P5A and P5A C12/H2 are heterodimeric variants of 5A and are synthesized by introducing two amino acids with antioxidant potential: cysteine and histidine.

### ELK-Based Peptides

In some embodiments, the peptide may comprise the monomeric form of the ELK peptide having the amino acid sequence EKLKELLEKLLEKLKELL (SEQ ID NO: 6). The ELK peptide contains only a combination of 3 amino acid residues: negatively charged glutamic acid, hydrophobic leucine, and positively charged lysine. In some embodiments, the peptide may be the homodimeric peptide having two identical copies of the ELK monomeric peptides coupled through a linker. The linker may be proline or alanine. The homodimeric peptide consists of two identical canonical type A amphipathic α helices with the hydrophobic interface turned by 180° and neutral net charge. In some embodiments, the peptide may be a homodimeric peptide having two identical copies of modified ELK monomeric peptides coupled through a linker, or a heterodimeric peptide having two different modified ELK monomeric peptides coupled through a linker. In those embodiments, the linker may be proline or alanine. In some embodiments, the peptide consists of L-amino acids.

In some embodiments, the peptide has the amino acid sequence shown in Table 3. FIG. 1 shows the comparative structure analyses of ELK (dimer) and ELK-B. ELK-B3 comprises a modified ELK peptide with a 2500 bigger hydrophobic phase and +4 net charge.

TABLE 3

| | | SEQ |
|---|---|---|
| Peptide | | ID |
| Name | Amino Acid Sequence | NO: |
| ELK | EKLKELLEKLLEKLKELL | 6 |
| (monomer) | | |
| ELK | EKLKELLEKLLEKLKELLP | 7 |
| (dimer) | EKLKELLEKLLEKLKELL | |
| ELK-B | EKLLELLKKLLELLKKLLP | 8 |
| | EKLLELLKKLLELLKKLL | |
| ELK-B2 | EKLKELLEKLLELLKKLLP | 9 |
| | EKLKELLEKLLELLKKLL | |
| ELK-C | EELKEKLEELKEKLEEKLP | 10 |
| | EELKEKLEELKEKLEEKL | |
| ELK-C1 | EELKAKLEELKAKLEEKLP | 11 |
| | EELKAKLEELKAKLEEKL | |
| ELK-C3 | EKLKELLEKLKAKLEELLP | 12 |
| | EKLKELLEKLKAKLEELL | |
| ELK-C4 | EKLKAKLEELKAKLEELLP | 13 |
| | EKLKAKLEELKAKLEELL | |
| ELK-D | EKLKALLEKLLAKLKELLP | 14 |
| | EKLKALLEKLLAKLKELL | |
| ELK-D2 | EKLKELLEKLLAKLKELLP | 15 |
| | EKLKELLEKLLAKLKELL | |
| ELK-E | EWLKELLEKLLEKLKELLP | 16 |
| | EWLKELLEKLLEKLKELL | |
| ELK-F | EKFKELLEKFLEKFKELLP | 17 |
| | EKFKELLEKFLEKFKELL | |
| ELK-F2 | EKFKELLEKLLEKLKELLP | 18 |
| | EKFKELLEKLLEKLKELL | |
| ELK-G | EELKELLKELLKKLEKLLP | 19 |
| | EELKELLKELLKKLEKLL | |
| ELK-H | EELKKLLEELLKKLKELLP | 20 |
| | EELKKLLEELLKKLKELL | |
| ELK-I | EKLKELLEKLLEKLKELLA | 21 |
| | EKLKELLEKLLEKLKELL | |
| ELK-J | EKLKELLEKLLEKLKELLA | 22 |
| | AEKLKELLEKLLEKLKELL | |
| ELK-K | DWLKAFYDKVACKLKEAFP | 23 |
| | DWAKAAYNKAAEKAKEAA | |
| ELK-L | DHLKAFYDKVAEKLKEAFP | 24 |
| | DWAKAAYDKAAEKAKEAA | |
| ELK-A2 | EKLKAKLEELKAKLEELLP | 25 |
| | EKAKAALEEAKAKAEELA | |
| ELK-AS | EKLKAKLEELKAKLEELLP | 26 |
| | EHAKAALEEAKCKAEELA | |
| ELK-B- | EKLLELLKKLLELLKKLLP | 30 |
| P-18A | DWLKAFYDKVAEKLKEAF | |

### ELR-Based Peptides

In some embodiments, the peptide may comprise the monomeric form of the ELR peptide having the amino acid sequence ERLLELLRRLLELLRRLL (SEQ ID NO: 27). The ELR-based peptides, including the ELR dimer (SEQ ID NO: 28) have been shown to exhibit potent anti-inflammatory properties. The ELR-based peptides, including ELR dimer (SEQ ID NO: 28) and ELR-B-P-18A (SEQ lID NO: 30), have also been shown to exhibit anti-pain and anti-swelling effects in CFA models of inflammatory pain. In some embodiments, the peptide may be the homodimeric peptide having two identical copies of the ELR monomeric peptides coupled through a linker. The linker may be proline. In some embodiments, the peptide may be a heterodimeric peptide having the ELR monomeric peptide or a modified ELR monomeric peptide, coupled to the 18A peptide through a linker. The linker may be proline. In some embodiments, the peptide consists of L-amino acids.

In some embodiments, the peptide has the amino acid sequence shown in Table 4.

TABLE 4

| Peptide | | SEQ |
|---|---|---|
| Name | Amino Acid Sequence | ID NO: |
| ELR | ERLLELLRRLLELLRRLL | 27 |
| (monomer) | | |
| ELR-B | ERLLELLRRLLELLRRLL | 28 |
| (dimer of | PERLLELLRRLLELLRRL | |
| ELR) | L | |
| ELR-P- | ERLLELLRRLLELLRRLL | 29 |
| 18A | PDWLKAFYDKVAEKLKEAF | |

In addition to the peptides described above, peptides of the present disclosure also encompass those peptides having a common biological activity and/or structural domain and having sufficient amino acid identity (homologues) as defined in this disclosure. These homologues can be from either the same or different species of animal, preferably from mammals, more preferably from rodents, such as mouse and rat, and most preferably from human. Preferably, they exhibit at least one structural and/or functional feature of apoA-I, and are preferably capable of treating conditions characterized by acute or chronic pain, for example by acting as antagonists of the activity of the HMGB1, HSP60, and/or SAA receptor. Such modifications include amino acid substitution, deletion, and/or insertion. Amino acid modifications can be made by any method known in the art and various methods are available to and routine for those skilled in the art.

In making amino acid substitutions, generally the amino acid residue to be substituted can be a conservative amino acid substitution (i.e., “substituted conservatively”), for example, a polar residue is substituted with a polar residue, a hydrophilic residue with a hydrophilic residue, hydrophobic residue with a hydrophobic residue, a positively charged residue with a positively charged residue, or a negatively charged residue with a negatively charged residue. Moreover, generally, the amino acid residue to be modified is not highly or completely conserved across species and/or is critical to maintain the biological activities of the peptide and/or the protein it derives from.

Furthermore, the present disclosure also encompasses variants and derivatives of the peptides of the invention. For example, but not by way of limitation, derivatives may include peptides or proteins that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, etc. Non-limiting examples of variants may include peptides that have been conservatively substituted by replacing a certain number of hydrophobic amino acid residues with other hydrophobic amino acid residues. Additionally, the variants and derivatives may contain one or more non-classical amino acids.

### Synthesis and Preparation of SAHPs

Peptides of the present disclosure may be directly synthesized in any appropriate way known to those of ordinary skill in the art. For example, but not by way of limitation, peptides of the present disclosure may be synthesized by a solid-phase procedure, such as those disclosed in Fairwell, T., et al., Proc. Natl. Acad. Sci. USA, 84:4796-4800 (1987) and Merrifield, R. B., Adv. Enzymol. Relat. Areas Mol. Biol., 32:221-296 (1969), each of which is incorporated by reference in its entirety in this disclosure.

### Pharmaceutical Compositions

SAHPs of the present disclosure are non-opioid, non-steroid, non-COX inhibitor compounds. The novel peptides offer a clear advantage over opiates in that they are not addictive, which is an important consideration against the backdrop of the ongoing opioid crisis. Further, peptides of the present disclosure are shown to be well tolerated and avoids adverse side effects such as skin irritation and liver toxicity and injury.

Peptides of the present disclosure can be used to prepare a pharmaceutical composition effective for treating conditions characterized by acute or chronic pain, including, but not limited to, sport-related joint pain, rheumatoid arthritis, burn-related pain, neuropathy including diabetic neuropathy, orthopedic pain, phantom pain in amputees and foot neuro-degenerative syndrome. Peptides of the present disclosure can also be used to prepare a pharmaceutical composition effective for treating conditions characterized by neuroinflammation.

The present disclosure provides a pharmaceutical composition comprising a therapeutically effective amount of a peptide of the invention. The peptides (also referred to in this disclosure as “active compounds”) of the invention can be incorporated into pharmaceutical compositions suitable for administration. Such compositions typically comprise the peptide and a pharmaceutically acceptable carrier.

The pharmaceutical composition may comprise at least one peptide selected from the group consisting of the 18A-based peptides. The pharmaceutical composition may comprise at least one of the longer and shorter analogs of the 18A-based peptides, including examples listed in Tables 2 to 4: at least one peptide selected from the group consisting of the ELK-based peptides, at least one peptide selected from the group consisting of the ELR-based peptides, and/or a combination thereof. In some embodiments, the pharmaceutical composition comprises the L37pA peptide having the sequence DWLKAFYDKVAEKLKEAFPDWLKAFYDKVAEKLKEAF (SEQ ID NO: 2). In some embodiments, the pharmaceutical composition comprises the ELR-B-P-18A peptide having the sequence EKLLELLKKLLELLKKLLPDWLKAFYDKVAEKLKEAF (SEQ ID NO: 30). In some embodiments, the pharmaceutical composition comprises a peptide that consists of L-amino acids.

As used in this application, “pharmaceutically acceptable carrier” refers to those components in the particular dosage form employed, which are considered inert and are typically employed in the pharmaceutical arts to formulate a dosage form containing a particular active compound. The pharmaceutically acceptable carrier includes any and all solvents, dispersion media, coatings, antibacterial and antifungal agents, isotonic and absorption delaying agents, and the like, that is compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A peptide of the present disclosure can be used as a monotherapy or in combination with other therapeutic agents. As a non-limiting example, the peptide may be used in conjunction with another pain antagonist or analgesic such as non-steroidal anti-inflammatory drugs (NSAIDs) and opioid analgesics. By way of example only, the therapeutic effectiveness of one of the peptides described in this disclosure may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit experienced by a patient may be increased by administering one of the peptides of the present disclosure with another therapeutic agent (which also includes a therapeutic regimen) that also has therapeutic benefit. In any case, regardless of the disease or condition being treated, the overall benefit experienced by the patient may be synergistic of the multiple therapeutic agents or the patient may experience a synergistic benefit. Where the peptides of the present disclosure are administered in conjunction with other therapies, dosages of the co-administered therapeutic agents will of course vary depending on the type of co-drug employed, on the specific drug employed, on the disease or condition being treated and so forth. In addition, when co-administered with one or more biologically active agents, the peptide of the present disclosure may be administered either simultaneously with the biologically active agent(s), or sequentially. If administered simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If administered sequentially, the attending physician will decide on the appropriate regimen. In addition, the combination methods, compositions and formulations are not to be limited to the use of only two agents. Multiple therapeutic combinations are envisioned.

As used in this application, “therapeutically effective amount” of a peptide is the amount that effectively achieves the desired therapeutic result in the subject. Such amounts may be initially determined by knowledge in the art, by conducting in vitro tests, and/or by conducting metabolic studies in healthy experimental animals or during clinical trials. Naturally, the dosages of the various peptides of the present disclosure will vary somewhat depending upon the host treated, the particular mode of administration, among other factors. Those skilled in the art can determine the optimal dosing of the peptide of the present disclosure selected based on clinical experience and the treatment indication.

The pharmaceutical composition according to the present disclosure is configured to facilitate administration of a peptide to a subject. A peptide of the present disclosure, or a pharmaceutical composition containing the peptide, can be administered according to any suitable method or route, including, but not limited to, intranasal, intramuscular, intratracheal, subcutaneous, intradermal, transdermal, sublingual, topical application, intravenous, ocular (e.g., topically to the eye, intravitreal, etc.), rectal, nasal, oral, topical administration, and other enteral and parenteral routes of administration.

Methods of administering a peptide of the present disclosure through the skin or mucosa include, but not limited to, topical application of a suitable pharmaceutical preparation, transdermal transmission, injection and epidermal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF; Parsippany, N.J.) or phosphate buffered saline (PBS). The composition must be sterile and should be sufficiently fluid to allow easy injectability with a syringe. The composition must be stable under the conditions of manufacture and storage and must be preserved against the contamination by microorganisms such as bacteria and fungi. The suitable carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by using a coating such as lecithin and by using appropriate surfactants. To prevent contamination, the composition may comprise a suitable antibacterial and/or antifungal agent. In some embodiments, the composition may comprise isotonic agents such as sugars and sodium chloride. In some embodiments, the composition may comprise an agent which delays absorption, for example, aluminum monostearate and gelatin to prolong absorption of the injected composition.

Sterile injectable solutions may be prepared by incorporating the peptide of the present disclosure in the therapeutically effective amount in an appropriate solvent with one or a combination of ingredients enumerated above, as required, followed by filtered sterilization. Generally, dispersions are prepared by incorporating the peptide into a sterile vehicle which contains a basic dispersion medium and the required other ingredients from those enumerated above.

In some embodiments, the pharmaceutical composition is formulated for a topical application, an intravenous administration, or an intramuscular injection. In some embodiments, the topical formulation is a lotion or a hydrogel. In some embodiments, the topical formulation may comprise an amount of the peptide sufficient to administer from about 4 to about 10 μg peptide per single skin treatment of from 20 to 50 μl of the topical formulation. In some embodiments, the topical formulation composition may comprise from 0.1 to 1.0 μg/μl of the peptide of the present disclosure. In some embodiments, the topical formulation may comprise 0.001 to 5 μg/μl of the peptide of the present disclosure. In some embodiments, the topical formulation may comprise 0.2 μg/μl or higher of the peptide of the present disclosure. In formulating the topical application, the volume of the topical formulation should also be considered. The full range of effective doses as evaluated by additional animal studies and/or clinical trials may be broader than the doses indicated above.

When the pharmaceutical composition is formulated for an intramuscular injection, the formulation may comprise an amount of the peptide sufficient to administer about 20 g of the peptide per injection at a dose of 2 μl/g of the subject's weight. In some embodiments, the intramuscular injection formulation may comprise 0.001 to 100 mg/ml of the peptide of the present disclosure. In some embodiments, the composition may be injected intramuscularly, for example to the thigh muscle, at a daily dose of 0.5 mg/kg to 100 mg/kg. The full range of effective doses as evaluated by additional animal studies and/or clinical trials may be broader than the doses indicated above.

The present disclosure provides methods for preparing pharmaceutical compositions containing a peptide of the invention. Such compositions can further include additional active agents. Thus, the present disclosure further provides methods for preparing a pharmaceutical composition by formulating a pharmaceutically acceptable carrier with a peptide of the invention and one or more additional active compounds.

When the pharmaceutical composition is formulated for an intravenous injection, the formulation may be administered at a dosage of 0.5 mg/kg to 100 mg/kg. The intravenous injection may be administered to, for example, the jugular vein.

With respect to the above-discussed administration routes, it is believed that subjects will tolerate a higher concentration of the peptides via topical administration as compared to intravenous or intramuscular administration.

### Treatment Methods

Surprisingly, the novel SAHP apoA-I mimetic peptides described in this application can be used for treatment of chronic and acute pains, including inflammatory and neuropathic pains. The peptides provide potent and effective pain-killing effects for the treatment and management of chronic and acute pains. The peptides can bind several receptors in vitro, including CD36, CLA-1, CLA-2 and can influence TLR activity. Not only has the efficaciousness of the peptides be shown in vitro using cell culture models, but the acute and chronic analgesic effects of the peptides have also been shown through in vivo studies in animal models.

The present disclosure thus provides methods for treatment of various conditions, especially conditions characterized by chronic or acute pain and conditions characterized by neuroinflammation. In some embodiments, the methods involve administering to a subject in need thereof (for example, a human or a non-human mammal) a therapeutically effective amount of a peptide of the present disclosure, or a pharmaceutical composition comprising a therapeutically effective amount of the peptide. The peptide may comprise at least one of the 18A-based peptides, at least one of the ELK-based peptides, at least one of the ELR-based peptides, and/or a combination thereof.

In some embodiments, the peptide is administered topically, intravenously, or intramuscularly (for example, by injection).

The “therapeutically effective amount” is as defined above. For intravenous administration, the peptide may be administered at doses of 0.5 mg/kg to 100 mg/kg. In some embodiments, the peptide may be administered at a dose of 10 mg/kg, 15 mg/kg, or 25 mg/kg. The full range of effective doses and frequency of application may be broader than doses indicated above.

In some embodiments, the peptide is formulated into a topical formulation. When applied topically, an amount of from about 4 to about 10 μg peptide may be administered per single skin treatment of from 20 to 50 μl of the topical formulation. In some embodiments, the peptide is formulated into an intramuscular injection formulation. When injected intramuscularly, the peptide may be administered at a dose of 2 μl/g and an amount of about 20 μg of the peptide may be administered per injection. The full range of effective doses as evaluated by additional animal studies and/or clinical trials may be broader than the doses indicated above.

In some embodiments, the peptide is administered once daily. The peptides of the present disclosure are able to cause sustained pain-relieving effect when administered repeatedly, once-a-day in mouse models of Complete Freund's Adjuvant (CFA) and neuropathic pain. In addition, the same daily treatment is highly effective in a limited study of patients with chronic neuropathic and orthopedic pain. In some embodiments where the peptide is formulated into a topical application, the formulation may be administered twice a day. The frequency of administration may be altered by additional animal studies or by experience gained during clinical trials.

In some embodiments, the peptide of the present disclosure is used in the treatment of pathologies that include, but are not limited to, sport-related joint pain, rheumatoid arthritis, burn-related pain, neuropathy including diabetic neuropathy, orthopedic pain, phantom pain in amputees and foot neuro-degenerative syndrome.

A peptide of the present disclosure may be used in combination with procedures that may provide additional or synergistic benefit to the patient. A peptide of the present disclosure, whether as monotherapy or in combination therapy, may be administered before, during or after the occurrence of the disease or condition to be treated, and the timing of administering the composition containing the peptide can vary. Thus, a peptide may be administered to a subject during or as soon as possible after the onset of the symptoms. A peptide is preferably administered as soon as is practicable after the onset of the disease or condition enumerated in the present disclosure is detected or suspected, and for a length of time necessary for the treatment of the disease. The length of treatment can vary for each subject, and the length can be determined using known criteria. Administration can be acute (for example, of short duration (e.g., single administration, administration for one day to one week)), or chronic (for example, of long duration, (e.g., administration for longer than one week, from about 2 weeks to about one month, from about one month to about 3 months, from about 3 months to about 6 months, from about 6 months to about 1 year, or longer than one year)).

The mechanism of the peptides of the present disclosure is not well-understood. The applicant believes that the combination of hydrophobic domains and helical structures, which cause not only association with HDL promoting cholesterol esterification and accelerates transport to the liver for eventual excretion to the bile, but which also results in a direct specific interaction of SAHP with class B scavenger receptors including SR-BI, SR-BII, and CD36. This class of SR-B has been demonstrated to participate in an innate immune response by recognizing and mediating downstream signaling and clearance of a number of PAMPs and DAMPs, including thromboplastin, acute phase proteins, SAA, heat shock proteins such as HSP60 and GRP94, as well as HMGB1. The binding motif in number of SR-B interacting DAMPs is the presence of amphipathic helical sequence in the structure. Such a structure is represented in the novel SAHPs of the present disclosure. The amphipathicity of these SAHPs allows them to form oligomers with hydrophobic amino acids facing core and hydrophilic water phase. The applicant has observed that the combination of hydrophobic and hydrophilic domains in the SAHPs of the present disclosure have a high affinity toward SR-B receptor interaction, making them highly effective competitors against other DAMPs and PAMPs. In addition to this ligand-receptor interaction, the SAHPs of the present disclosure can directly associate with amphipathic helix containing proteins and lipoprotein particles. This effect has been shown for HDL and L37pA, which neutralize LPS by direct association and inclusion of the PAMP into HDL particles or L37pA quadro/tetrameric complexes/micelles. L37pA, as well as other SAHPs such as ELK-B disclosed in this application, can directly compete with DAMPs and PAMPs for CD36 to prevent downstream signaling. The SAHPs can also bind the DAMPs and/or PAMPs, neutralize proinflammatory activity, and serve as a bridge targeting SAHP oligomers or SAHP-phospholipid complexes/micelles bound cargo to SR-B receptors for DAMP/PAMP clearance helping to escape interaction with other innate immune response receptors such as TLRs.

In addition, the mechanism by which the SAHPs of the present disclosure alleviates pain is not fully elucidated. The applicant hypothesizes that, as one potential mechanisms, sub-chronic damage of neurons and surrounding tissue leads to local release of DAMPs that perpetuates inflammation to induce further increased release of DAMPS, pain-inducing chemokines, escalating pain sensation (excitation of pain neuronal activity) and/or hypersensitivity of pain receptors, and it is believed that the SAHP peptides of the present disclosure relieve pain sensation and pain genesis by neutralizing DAMPs and inhibiting CD36-mediated DAMP/PAMP uptake. As such, the SAHP peptides of the present disclosure may potentially inhibit pain signaling caused by a broad range of DAMP and PAMP families of damaging mediators. Several small molecule inhibitors have been proposed that bind pain-inducing mediators and other DAMPs and that are based on DAMP ligand binding and neutralization principle. On the other hand, the SAHP peptides of the present disclosure consider three independent mechanisms of peptide action: first, via direct binding/neutralization of DAMP ligand; second, peptide binding to and inhibition of CD36 receptor pro-inflammatory activity; and three, via potential antagonism of pain-sensing and pain-conducting signaling.

In addition to pain relief and management, the biological activities of the peptides may also facilitate accelerated nerve regeneration and wound healing (mitigation of local inflammation). The peptides may also be used in burn treatment to both treat pain and to facilitate healing of the burn wound. The peptides of the present disclosure are stable and may be used as therapeutic agents for the treatment of a broad range of diseases beyond those described in this application.

As used in this application, “treat” and all its forms and tenses (including, for example, treat, treating, treated, and treatment) refer to therapeutic treatment and/or prophylactic or preventative treatment. Those in need of treatment include those already with a pathological condition enumerated in the present disclosure as well as those in which the pathological condition is to be prevented. For example, “treat” means alter, apply, effect, improve, care for or deal with medically or surgically, ameliorate, cure, stop and/or prevent an undesired biological (pathogenic) process. Those of ordinary skill in the art are aware that a treatment may or may not cure.

### EXAMPLES

The following examples are offered to illustrate the present invention and to assist one of ordinary skill in the relevant art in making and using the present invention. The examples are not intended to limit the scope of the present invention.

### Example 1: Testing of Analgesic Effects of L37pA Peptide in Complete Freund's Adjuvant (CFA) Animal Model of Chronic Inflammatory Pain

This example demonstrates the chronic analgesic effect of the L37pA peptide in vivo using an animal model of chronic inflammatory pain. The L37pA peptide having the sequence DWLKAFYDKVAEKLKEAFPDWLKAFYDKVAEKLKEAF (SEQ ID NO: 2) was synthesized by a solid-phase procedure. An inactive L37pA peptide where three L-lysine's in each A18 were replaced with D-lysine's disrupting the helical L37pA structure, L3D-37pA (L3D), was synthesized as a negative control. The L37pA and L3D-37pA peptides were formulated into both an intramuscular injectable preparation and a hydrogel or lotion preparation. The intramuscular (IM) injectable formulation contains 0.5 μg/μl of the peptide, with an injection dose of 2 μl/g mouse weight, which equals approximately 20 μg peptide per injection. The hydrogel and lotion formulations each contains 0.2 mg/ml of the peptide. 20-50 μl of the formulations are applied, which is approximately 4-10 μg peptide per single skin treatment. Topical hydrogel and lotion formulations containing a vehicle without any peptide were prepared as additional negative controls.

### Intramuscular Injection

In this experiment, the peptide was administered via intramuscular injections. The first group of mice (“CFA group”) were injected with 30 μl of Complete Freund's Adjuvant (CFA) in the plantar surface of the left hind paw. The second group of mice (“Vehicle Group”) were injected with Vehicle (saline) instead of CFA. For each of the CFA and Vehicle Groups, the mice were then injected once a day with the following formulations, with “n” representing the number of mice receiving each formulation:

| | |
|---|---|
| Vehicle/IM injection L3D-37pA | n = 4 |
| Vehicle/IM injection L37pA | n = 4 |
| CFA/IM injection L3D-37pA | n = 4 |
| CFA/IM injection L37pA | n = 4 |

Thermal hyperalgesia was performed before the CFA injection. The measurement of baseline thermosensitivity was actually carried out before time 0, but it was assumed that the baseline remained the same at time 0. Injection of CFA in the paw and the intramuscular injection of peptide were performed at the same time. Injections of CFA and peptides were performed in left paws. The right paws of the mice remained intact and were used for comparison.

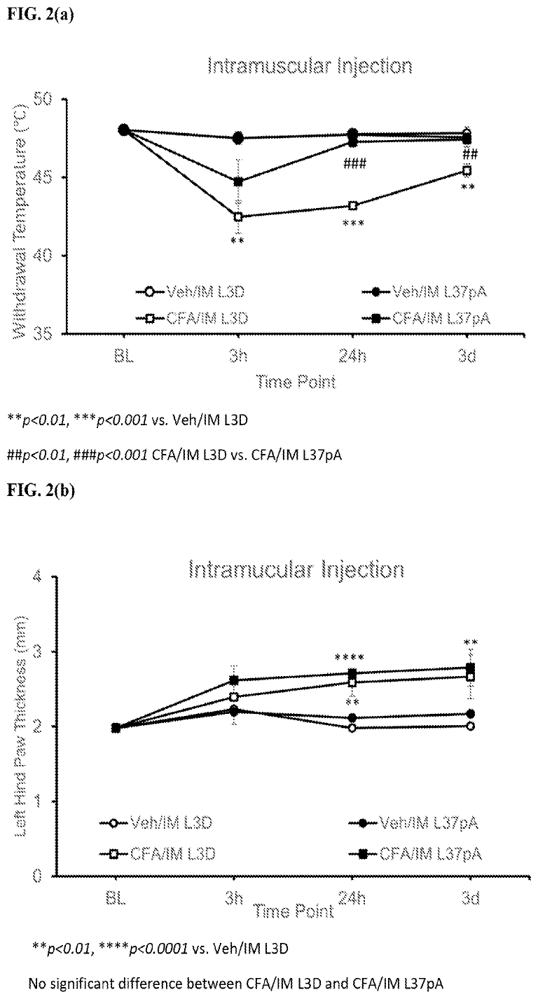

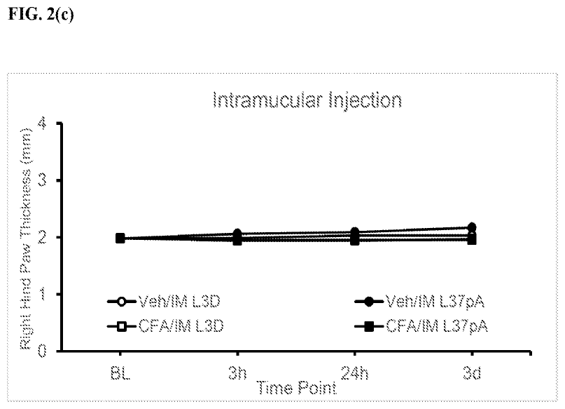

After administering the L37pA or L3D-37pA peptide, the thermal hyperalgesia test was performed for 30 minutes at 3, 24, and 72 hours post CFA-injection. In the thermal hyperalgesia test, an incremental hot plate with an automatic cut-off temperature of 50° C. was used to induce the nocifensive behaviors of licking a hind paw to identify the thresholds for noxious heat and assess the thermal hyperalgesia. The temperature of the plate at the time when the licking occurred was recorded. The peptides were injected every single day and at 30 minutes prior to the behavior test. In addition, to investigate the effects of the peptides on inflammatory swelling caused by the CFA injection, the thicknesses of the left and right hind paws of the mice were measured post-peptide administration for signs of inflammation. The results are shown in FIGS. 2(a)-2(c). In the figures, *p≤0.05, **p≤0.01, and ***p≤0.001 show comparisons to the Vehicle/L3D-37pA controls at each timepoint.

### Topical Application

In this experiment, the peptide was administered topically once a day. The first group of mice (“CFA group”) were injected with 30 μl of Complete Freund's Adjuvant (CFA) in the plantar surface of the left hind paw. The second group of mice (“Vehicle Group”) were injected with Vehicle (saline) instead of CFA. For each of the CFA and Vehicle groups, the topical formulation was then administered to the mice according to the following experiment design:

| | |
|---|---|
| Vehicle/Topical L3D-37pA | n = 3 (all gel) |
| Vehicle/Topical L37pA | n = 3 (all gel) |
| CFA/Topical Vehicle | n = 4 (2 lotion, 2 gel) |
| CFA/Topical L3D-37pA | n = 3 (all gel) |
| CFA/Topical L37pA | n = 7 (3 lotion, 4 gel) |

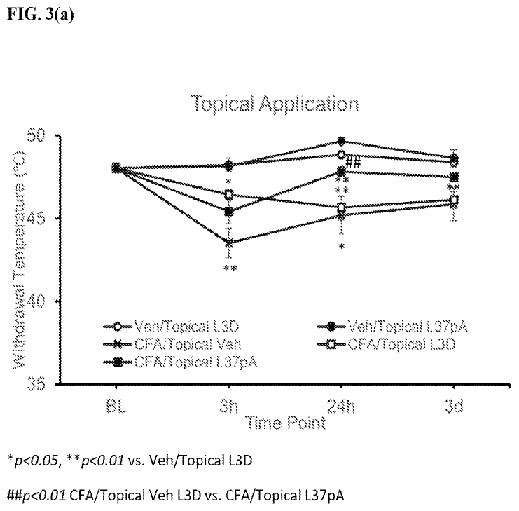

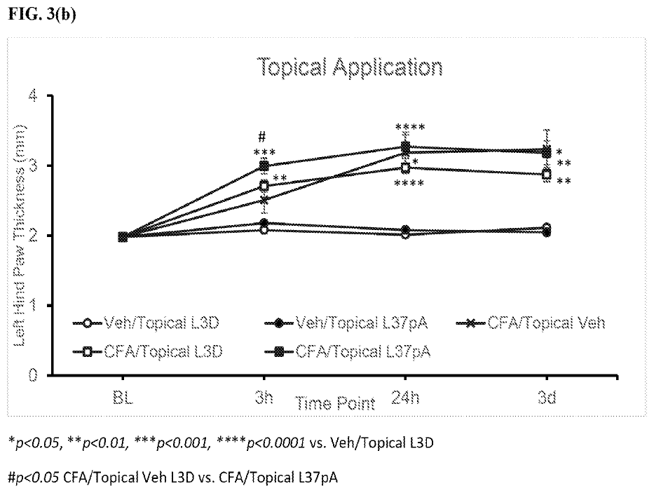

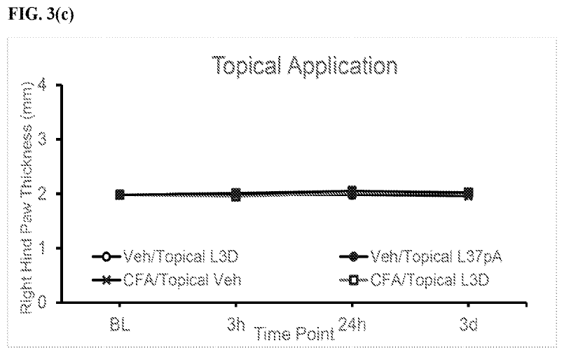

Baseline values for withdrawal latencies to thermal stimuli did not differ among five different test groups of mice, saline and L3D-L37 pA, saline and topical application of L37pA, CFA and vehicle, CFA and L3D-L37pA, and CFA and L37pA. The topical application of the peptides was carried out 21 hours after the saline or CFA injection and applied twice a day in the following days. In case of topical application of peptide, the peptides were applied 3 hours before the second measurement of thermosensitivity and applied twice a day afterwards. Thermal hyperalgesia was performed before administration and after administering the L37pA, L3D-37 pA, or vehicle, at 30 minutes after administration. The thermal hyperalgesia test was performed as described above. In addition, the thicknesses of the left and right hind paws of the mice were measured post-peptide administration for signs of inflammation. The results are shown in FIGS. 3(a)-3(c). In the figures, *p 0.05, **p 0.01, and ***p 0.001 show comparisons to the saline/L3D-37pA controls at each timepoint.

### Results

L37pA, inactive peptide L3D-L37pA, and vehicle were administered as an intramuscular injection (FIG. 2) or as a topical application in the form of hydrogel (FIG. 3) every consecutive day 30 minutes prior to behavioral test of CFA-injected or Vehicle-injected plantar surface of left hind paw. Pain was assessed as a decreased paw withdrawal temperature. Altered paw withdrawal effect in inflamed paws developed within 3 hours post-CFA injection (FIG. 2(a) and 3(a)). Importantly, topical application of the L37pA hydrogel formulation or local intramuscular injection returned the withdrawal temperature of inflamed paws to control levels measured in non-injected paw within 30 minutes after peptide application. This rapid analgesic effect of L37pA peptide application was observed in inflamed paws within 3 days post-CFA, while negative control L3D-L37pA peptide was without effect (FIGS. 2(a) and 3(a)). In contrast to pain relief, the inflammation sign (paw thickness) was not recovered to normal level upon short-term peptide application (FIGS. 2(b) and 3(b)). CFA-injected mice developed inflammatory sign in the form of increased paw thickness within 3 hours after CFA injection which peaked within 1 day (FIGS. 2(b) and 3(b)). Contralateral (non-injected) hind paw thickness remained unchanged (FIGS. 2(c) and 3(c)).

FIG. 2(a) shows that the intramuscular injection of peptide L37pA reduces inflammatory heat hyperalgesia. The test groups were: (1) saline injection in left paw, treatment with inactive peptide L3D-L37pA, (2) saline injection in left paw, treatment with active peptide L37pA, (3) CFA injection in left paw, treatment with inactive peptide L3D, and (4) CFA injection in left paw, treatment with active peptide L37pA. Baseline values for withdrawal latencies to thermal stimuli did not differ among the different groups of mice that were randomly allocated into four groups. In two groups of mice receiving saline injection in their left paw (designated as control groups without induced paw inflammation), there was no difference in their withdrawal latencies to thermal stimuli between the group that received the control peptide and the group that received L37pA at times 3 hours, 24 hours and 3 days after saline/intramuscular injection. The withdrawal latencies at these times remained about the same in both groups. However, in two groups of mice receiving CFA injection for 3 hours (CFA-inducedpaw inflammation), there was a significant reduction of withdrawal latencies and the reduction was even more exaggerated in the group receiving the control peptide L3D treatment compared to mice receiving L37pA. At 24 hours after the CFA/peptide co-treatment, the L37pA group had significantly increased withdrawal latencies to thermal stimuli in contrast to the L3D group which only showed a minimal increase. Three days after CFA/intramuscular peptide treatment, the withdrawal latencies to thermal stimuli in the L37pA group were same as the Sal groups, whereas the L3D group still showed a significant reduction of withdrawal latencies.

FIG. 2(b) shows the thickness of CFA-injected left paws in all four test groups of mice enumerated above. In two groups of mice receiving CFA treatment for 3 hours, the left paw showed increased thickness and slowly continued the trend at 24 hours and 3 days in both groups. There were no significant differences between the CFA groups receiving intramuscular injection of L3D or L37pA with respect to increased paw thickness upon CFA injection.

FIG. 2(c) shows that the thickness of untreated right paws in all four test groups of mice is similar at all the time points. In the two test groups injected with saline instead of CFA, mice only showed slight increase in their left paw thickness at 3 hours, but the paw thickness did not increase at 24 hours and 3 days. There were no significant differences between the group receiving intramuscular injection of L3D and that receiving L37pA.

FIG. 3(a) shows that the topical application of L37pA reduces heat hyperalgesia in the CFA model of inflammatory pain. Baseline values for withdrawal latencies to thermal stimuli did not differ among the five different test groups of mice, Vehicle and L3D, saline and topical application of L37pA, CFA and vehicle, CFA and L3D, and CFA and L37pA. The topical application of the peptides was carried out 21 hours after the Vehicle or CFA injection and applied twice a day in the following days. In mice receiving Vehicle injection in their left paw, no significant difference in their withdrawal latencies to thermal stimuli at different times. There were no significant differences between the two Vehicle groups that received the topical application of control peptide L3D and the group that received topical application of L37pA at 3 hours, 24 hours, and 3 days. However, in the three groups of mice receiving CFA injection, there was a significant reduction of withdrawal latencies 3h after the CFA treatment. At 24 hours and 3 days after the CFA treatment, the L37pA group had a significant increase of withdrawal latencies to thermal stimuli, while in the groups that received control peptide or vehicle, withdrawal latencies to thermal stimuli remained reduced with only slight increase.

FIG. 3(b) shows that the topical application of L37pA does not affect CFA-induced increase in left paw thickness. In the three groups of mice with CFA treatment, the left paw showed increased thickness at 3 hours, 24 hours, and 3 days. There was no significant difference between the groups receiving topical application of L3D, L37pA, or vehicle. In the two groups injected with saline instead of CFA, increase in their left paw thickness was negligible. There were no differences between the groups receiving topical L3D and L37pA. In the five groups of mice, the untreated right paws had the similar thickness at all the time points.

FIG. 3(c) shows that in the five test groups of mice, the untreated right paws had the similar thickness at all the time points.

Thus, as shown in the figures, the CFA model showed statistically significant reversal of pain sensation in response to stimuli by topical application of the L37pA peptide as hydrogel or lotion, and by local intramuscular injection. Formulations without the L37pA peptide or formulations containing inactive peptide analog (L3D-37pA) were used as negative controls and did not show any pain-reducing activities. The results showed that chronic administration of the L37pA peptide produces sustained pain-relieving effect.

In addition, the results show that the pain-reducing effects of the L37pA peptide are independent of its anti-inflammatory properties because the pain-reducing effects were detected as early as 30 minutes post-peptide administration and did not affect limb inflammatory swelling caused by injection of CFA.

Both L37pA concentrations and routes of administration caused statistically significant alleviation of pain as compared to control groups treated with vehicle (hydrogel or saline) or inactive peptide L3D-L37pA. Topical application caused significantly higher inhibitory effect on paw inflammation in CFA model by thickness than intramuscular injection. For the CFA model, repetitive intramuscular injections were performed up to 3 times (one injection daily), 30 minutes prior to pain test at 3 hours, 24 hours, and 72 hours after CFA injection. For the CFA model, repetitive skin applications were performed up to 3 times (one injection daily), 30 minutes prior to pain test at 3 hours, 24 hours, and 72 hours after CFA injection.

Moreover, up to 14 repetitive daily skin applications of vehicle (hydrogel) and L37pA peptide formulation were performed, and in all cases, no adverse effects (general toxicity, behavioral changes, weight loss, skin redness, skin erosions) were noted. Two weeks after the above-described experiments were stopped, the same mice were used in other experiments with intratracheal injection of LPS and HKSA. The mice still responded to the agents normally and those mice without LPS/HKSA treatment were “clean” as the naïve B6 mice as demonstrated by BAL analysis. Chronic topical administration of the L37pA peptide in the therapeutic range of doses does not induce skin irritation or other adverse side effects.

These data show that for the CFA model of chronic pain, the relieving effect of a single L37pA application 30 minutes before pain assessment was observed at different time points after pain initiation. Additionally, a cumulative pain-relieving effect of repetitive (daily) L37pA treatment was also observed. Therefore, mice receiving a daily L37pA skin application retain the pain alleviation effect for next few days, even if L37pA treatment was not performed prior to pain measurements.

### Example 2: Testing of Comparative Analgesic Effects of L37pA Peptide in Complete Freund's Adjuvant (CFA) Animal Model of Chronic Inflammatory Pain, Via Intramuscular Injection or Topical Administration

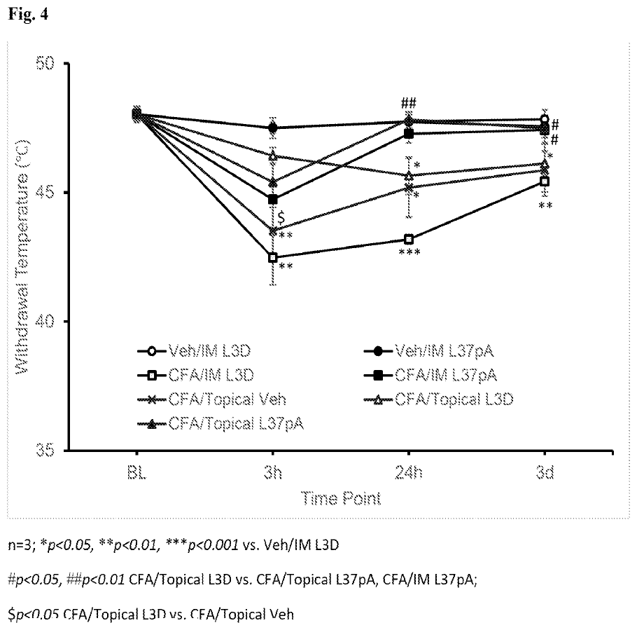

This example demonstrates the comparative effectiveness of L37pA by intramuscular injection and topical application. The applicant performed heat hyperalgesia tests similar to the manner discussed above in Example 1. In FIG. 4, six different test groups of mice were administered, a saline injection and inactive L3D-L37pA peptide intramuscular injection, a saline injection and the active L37pA peptide intramuscular injection, a paw CFA injection and inactive L3D-L37pA peptide intramuscular injection, a paw CFA injection and the active L37pA peptide intramuscular injection, a paw CFA injection and vehicle, and a paw CFA injection and a topical application of the active L37pA peptide. Shown in FIG. 4 are raw response temperatures at baseline, 3 h, 24 h, 3 days, and at 7 days after paw injection of CFA.; n=3; *p<0.01, #p<0.05.

As shown, the inactive L3D-37pA peptide did not reduce heat hyperalgesia at any time point, indicating that it was not protective against inflammatory heat hyperalgesia. On the other hand, the active L-37 pA peptide significantly reversed heat hyperalgesia (increased response temperature back toward baseline) at 24 h, 3 d, and 7 d in both the topical and intramuscular injection groups. The performance of the topical application and intramuscular injection groups were similar, indicating that these application routes may be substantially equally effective. It is noted that the analgesia effect of L37pA developed within minutes after topical application of L37pA, indicating that the SAHP can act directly at the level of nociceptor pattern recognition receptors.

### Example 3: Comparison of Analgesic Effects of Various Synthetic Amphipathic Helical Peptides in Complete Freund's Adjuvant (CFA) Animal Model of Chronic Inflammatory Pain, Via Topical Administration

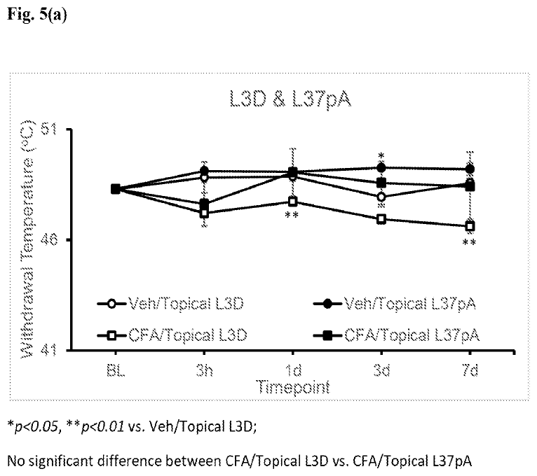

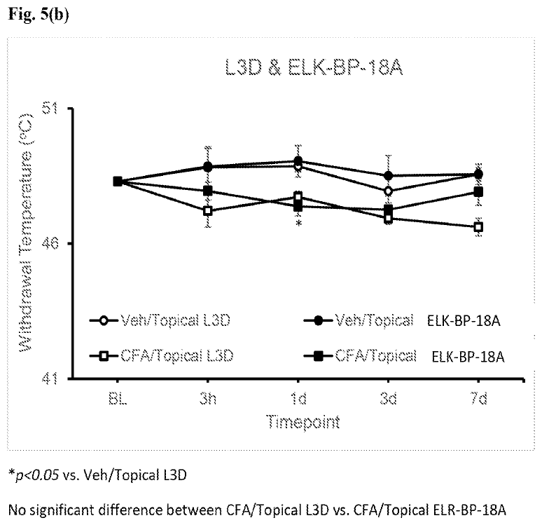

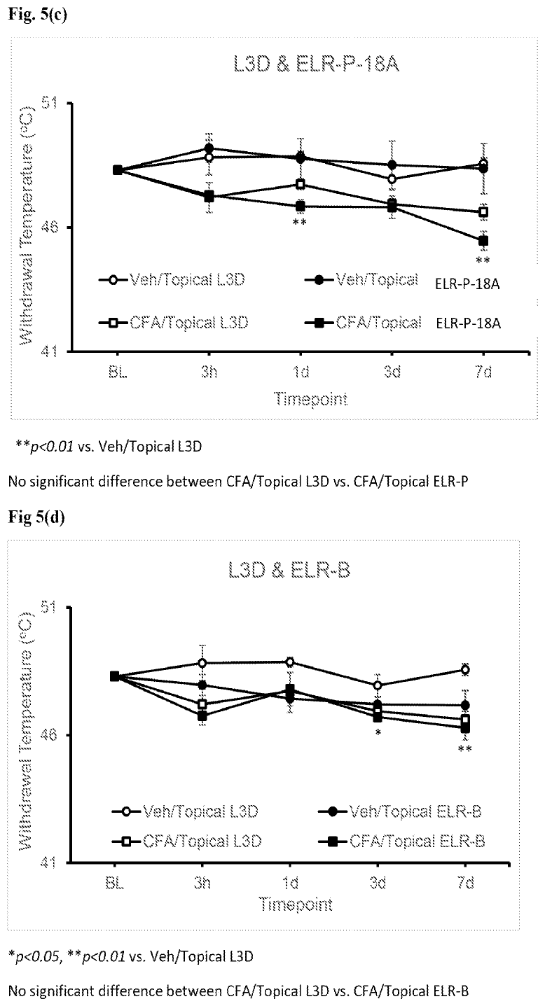

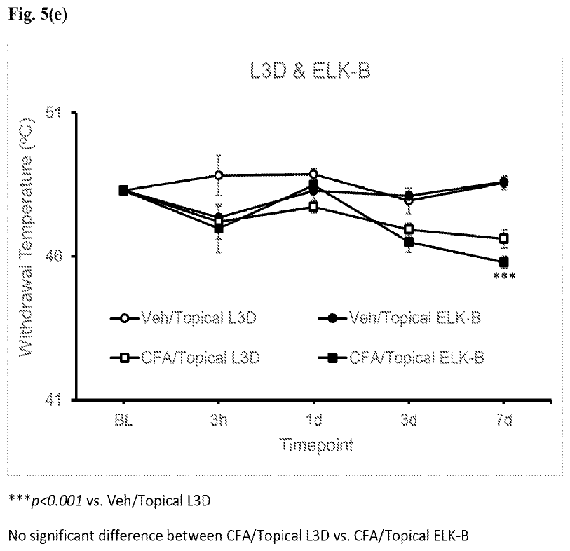

This example demonstrates the comparative chronic analgesic effect of various synthetic amphipathic helical peptides in addition to the L37pA peptide. The applicant performed heat hyperalgesia tests similar to the manner discussed above in Examples 1 and 2. In each of FIGS. 5(a) to 5(e), four different test groups of mice were administered a saline injection and a topical application of the peptide of interest, a saline injection and a topical application of the inactive L3D-L37pA peptide, a CFA injection and a topical application of the peptide of interest, and a CFA injection and a topical application of the inactive L3D-L37pA peptides. The peptides of interest were L37pA (SEQ ID NO: 2), ELK-BP-18A (SEQ ID NO: 30), ELR-P-18A (SEQ ID NO: 29), ELR-B (SEQ ID NO: 28), and ELK-B (SEQ ID NO: 8).

As shown in FIG. 5(a), L37pA improved heat hyperalgesia compared to the inactive L3D-L37pA peptide at all timepoints. As shown in FIG. 5(b), ELK-B-P-18A improved heat hyperalgesia compared to the inactive L3D-L37pA peptide at 3 hours, and particularly improved heat hyperalgesia at 7 days. As shown in FIG. 5(c), ELR-P-18A resulted in similar or worse heat hyperalgesia as compared to the inactive L3D-L37pA peptide. As shown in FIG. 5(d), ELR-B resulted in similar heat hyperalgesia compared to the inactive L3D-L37pA peptide. As shown in FIG. 5(e), ELK-B resulted in slightly improved heat hyperalgesia compared to the active L3D-L37pA peptide at 1 day, and similar heat hyperalgesia at other timepoints.

This data demonstrates that other synthetic amphipathic helical peptides besides L37pA may have a chronic analgesic effect. In particular, ELK-B-P-18A is a particularly promising peptide for an analgesic effect.

### Example 4: Testing of Analgesic Effects of L37pA Peptide in PSNL Animal Model of Neuropathic Pain Using Mechanical Allodynia

This example demonstrates the chronic analgesic effect of the L37pA peptide in vivo using a PSNL animal model of chronic neuropathic pain using mechanical allodynia as a measure. The L37pA peptide having the sequence DWLKAFYDKVAEKLKEAFPDWLKAFYDKVAEKLKEAF (SEQ ID NO: 2) was synthesized by a solid-phase procedure. An inactive L37pA peptide where three L-lysine's in each A18 were replaced with D-lysine's disrupting the helical L37pA structure, L3D-37pA (L3D), was synthesized as a negative control. The L37pA and L3D-37pA peptides were formulated into a hydrogel or lotion preparation. The hydrogel and lotion formulations each contains 0.2 mg/ml of the peptide. Topical hydrogel and lotion formulations containing a vehicle without any peptide were prepared as additional negative controls.

Mice were anesthetized by local anesthesia and analgesia using isoflurane, MARCAINE® and carprofen. A 1.5-cm longitudinal incision was made in the left hip and the proximal sciatic nerve was exposed. On the sham mouse, the skin incision was closed by sterile suture and Vetbond glue. On the PSNL mouse, before the closure of the skin, sterile suture was passed through the dorsal 1/3 of the sciatic nerve and tied for ligation. Mechanical allodynia was tested before surgery to establish a baseline (BL), at 7 days post operation (opt). Topical application was performed at day 8 post operation. The topical formulation was administered to the mice according to the following experiment design:

| | |
|---|---|
| Sham mouse/Topical Vehicle | n = 3 |
| Sham mouse/Topical L37pA | n = 3 |
| PSNL/Topical Vehicle | n = 3 |
| PSNL/Topical L37pA | n = 3 |

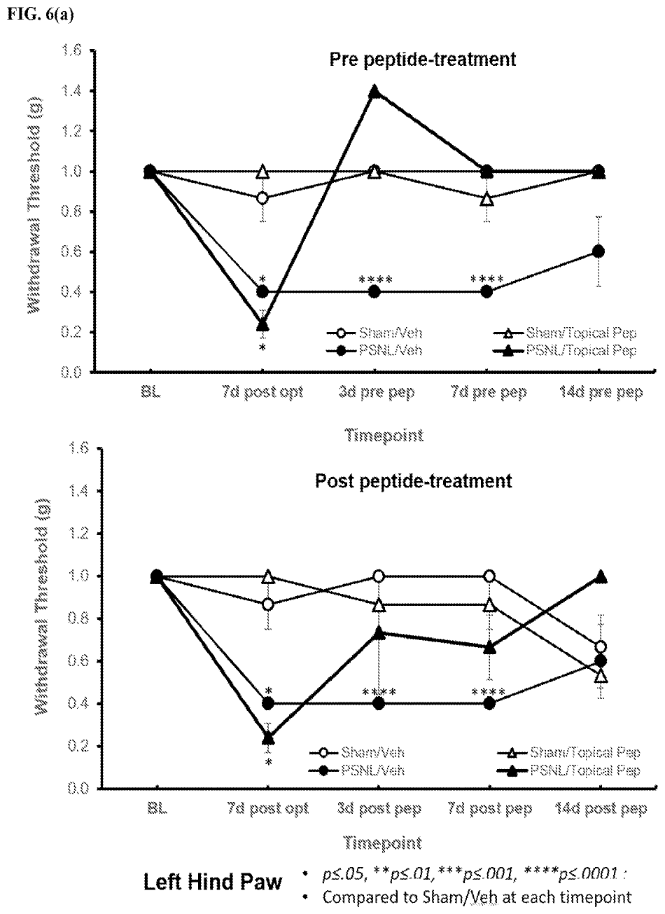

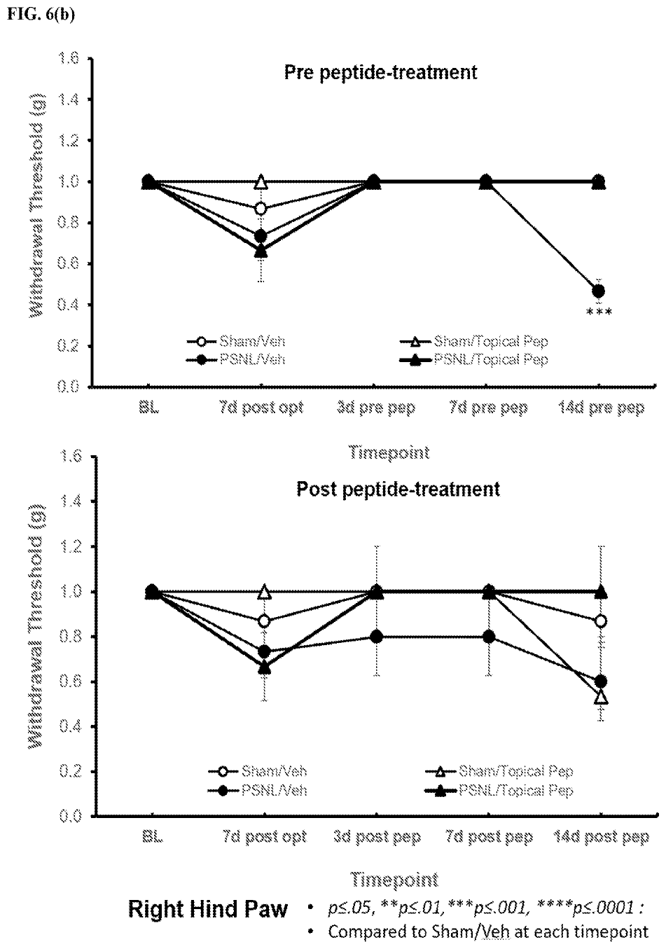

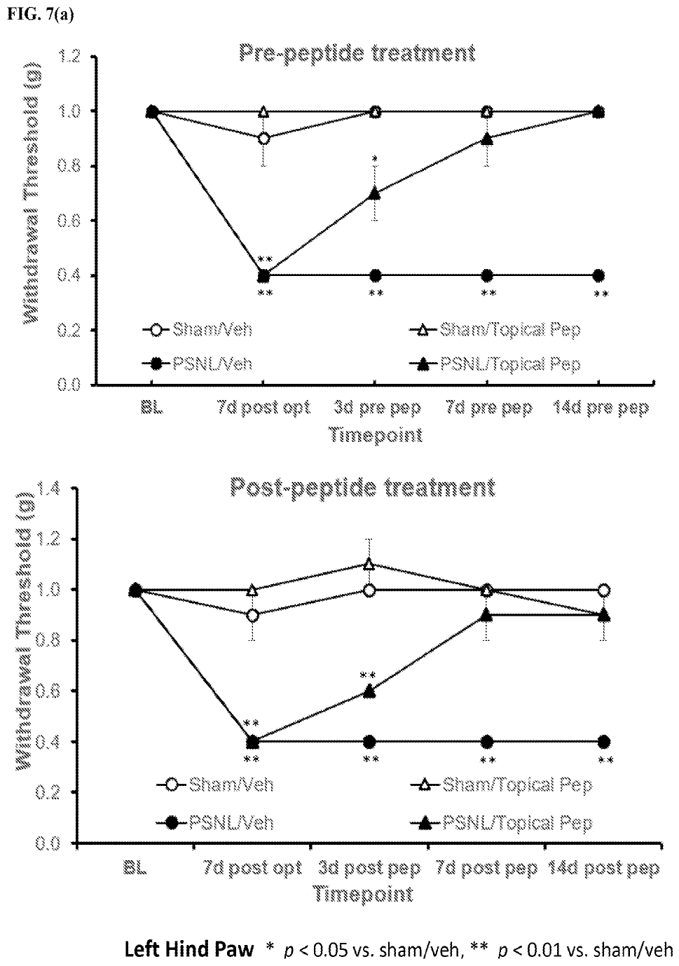

Mechanical allodynia was tested prior to topical application and at 30 minutes post-application at day 3, 7, 14 days post-topical application. To perform the mechanical allodynia test, von Frey filaments with incremental stiffness ranging from 0.04 g to 2.0 g were applied to the plantar surface of the hind paw to detect the hind paw's withdrawal from a mechanical allodynia. Threshold was defined as the filament with the lowest bending force that elicited at least 3 positive responses out of 5 trials. The results are shown in FIGS. 6(a)-6(b).

FIG. 6(a) shows that repetitive L37pA daily topical skin application reduces paw withdrawal threshold in PSNL model. In the top panel, left hind paw-operated mice were left to recover for 7 days. First pain assessment in all groups was performed at day 7 after surgery and served as “developed neuropathic pain” reference point. A topical application of hydrogel formulation containing L37pA or vehicle began at day 7 post-surgery and was performed daily in the next 14 days. The withdrawal threshold sensitivity was assessed after 3, 7 and 14 days of chronic peptide treatment, but 30 minutes before the regular daily peptide gel application (pre peptide-treatment).

In the bottom panel, in the same mice, the withdrawal threshold sensitivity was assessed after 3, 7 and 14 days of chronic peptide treatment, but 30 minutes after the regular daily peptide gel application (post peptide-treatment). Starting from day-3 of daily application, L37pA relieved the pain in PSNL mice to baseline levels registered in pre-operated and sham-operated mice, while control vehicle was without effect.

FIG. 6(b) shows that repetitive L37pA daily skin application in PSNL model—control tests. Paw withdrawal threshold remained unchanged in contralateral (non-operated) right hind paws 30 minutes before (top panel) and 30 minutes after (bottom panel) the regular daily L37pA peptide gel application.

As shown in the FIGS. 6(a) and 6(b), the PSNL model showed statistically significant reversal of pain sensation in response to stimuli by topical application of the L37pA peptide as hydrogel or lotion. Formulation without the L37pA peptide was used as a negative control and did not show any pain-reducing activities. The results showed that chronic administration of the L37pA peptide produces sustained pain-relieving effect.

In the PSNL model, repetitive skin application of L37 peptide after PSNL surgery caused sustained reduction of pain. The effect was observed after minimum 3 repetitive (daily) peptide applications. Testing pain 24 hours after the third peptide application showed statistically significant alleviation of neuropathic pain.

Moreover, up to 14 repetitive daily skin applications of vehicle (hydrogel) and L37pA peptide formulation were performed, and in all cases, no adverse effects (general toxicity, behavioral changes, weight loss, skin redness, skin erosions) were noted. Two weeks after the above-described experiments were stopped, the same mice were used in other experiments with intratracheal injection of LPS and HKSA. The mice still responded to the agents normally and those mice without LPS/HKSA treatment were “clean” as the naïve B6 mice as demonstrated by BAL analysis. Chronic topical administration of the L37pA peptide in the therapeutic range of doses does not induce skin irritation or other adverse side effects.

These data show that for the PSNL model of chronic pain, the relieving effect of a single L37pA application 30 minutes before pain assessment was observed at different time points after pain initiation. Additionally, a cumulative pain-relieving effect of repetitive (daily) L37pA treatment was also observed. Therefore, mice receiving a daily L37pA skin application retain the pain alleviation effect for next few days, even if L37pA treatment was not performed prior to pain measurements. See “pre-peptide treatment.”

### Example 5: Additional Testing of Analgesic Effects of L37pA Peptide in PSNL Animal Model of Neuropathic Pain Using Mechanical Allodynia

This example further demonstrates the chronic analgesic effect of the L37pA peptide in vivo using a PSNL animal model of chronic neuropathic pain using mechanical allodynia as a measure. As above, the L37pA peptide having the sequence DWLKAFYDKVAEKLKEAFPDWLKAFYDKVAEKLKEAF (SEQ ID NO: 2) was synthesized by a solid-phase procedure. An inactive L37pA peptide where three L-lysine's in each A18 were replaced with D-lysine's disrupting the helical L37pA structure, L3D-37pA (L3D), was synthesized as a negative control. The L37pA and L3D-37pA peptides were formulated into a hydrogel or lotion preparation. The hydrogel and lotion formulations each contains 0.2 mg/ml of the peptide. Topical hydrogel and lotion formulations containing a vehicle without any peptide were prepared as additional negative controls.

Mice were anesthetized by local anesthesia and analgesia using isoflurane, MARCAINE® and carprofen. A 1.5-cm longitudinal incision was made in the left hip and the proximal sciatic nerve was exposed. On the sham mouse, the skin incision was closed by sterile suture and Vetbond glue. On the PSNL mouse, before the closure of the skin, sterile suture was passed through the dorsal 1/3 of the sciatic nerve and tied for ligation. Mechanical allodynia was tested before surgery to establish a baseline (BL), at 7 days post operation (opt). Topical application was performed at day 8 post operation. The topical formulation was administered to the mice according to the following experimental design:

| | |
|---|---|
| Sham mouse/Topical Vehicle | n = 4 |
| Sham mouse/Topical L37pA | n = 4 |
| PSNL/Topical Vehicle | n = 4 |
| PSNL/Topical L37pA | n = 4 |

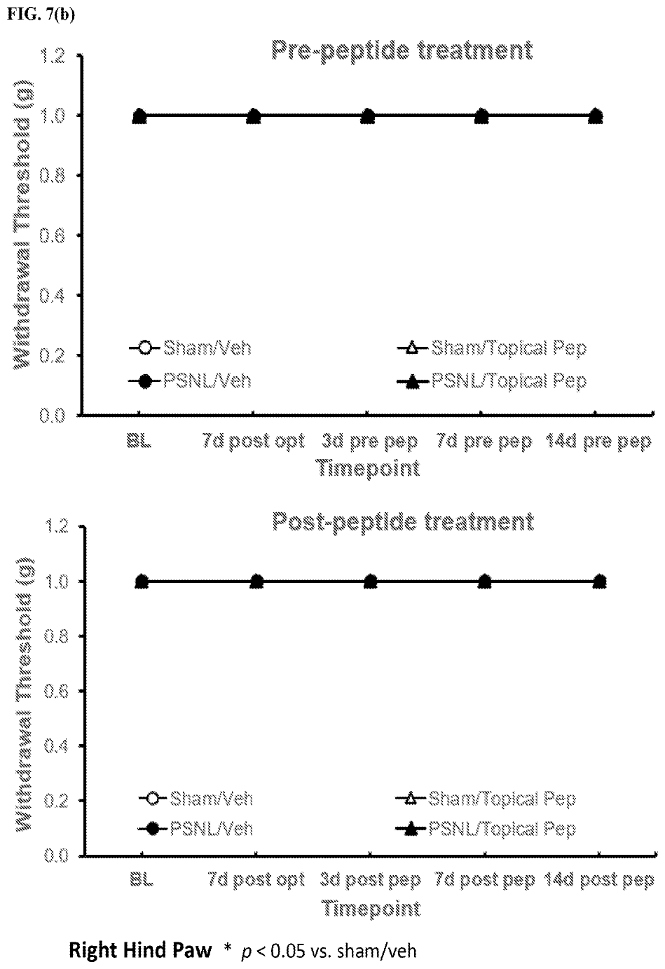

Mechanical allodynia was tested prior to topical application and at 30 minutes post-application at day 3, 7, 14 days post-topical application. The mechanical allodynia test was performed in the same manner as in Example 4, above. The results are shown in FIGS. 7(a) and 7(b).

FIG. 7(a) shows that repetitive L37pA daily topical skin application gradually reduces paw withdrawal threshold in PSNL model. In the top panel, left hind paw-operated mice were left to recover for 7 days. First pain assessment in all groups was performed at day 7 after surgery and served as “developed neuropathic pain” reference point. A topical application of hydrogel formulation containing L37pA or vehicle began at day 7 post-surgery and was performed daily in the next 14 days. The withdrawal threshold sensitivity was assessed after 3, 7 and 14 days of chronic peptide treatment, but 30 minutes before the regular daily peptide gel application (pre peptide-treatment).

In the bottom panel, in the same mice, the withdrawal threshold sensitivity was assessed after 3, 7 and 14 days of chronic peptide treatment, but 30 minutes after the regular daily peptide gel application (post peptide-treatment). Starting from day-3 of daily application, L37pA gradually relieved the pain in PSNL mice to baseline levels registered in pre-operated and sham-operated mice, while control vehicle was without effect.

FIG. 7(b) shows repetitive L37pA daily skin application in PSNL model—control tests. Paw withdrawal threshold remained unchanged in contralateral (non-operated) right hind paws 30 minutes before (top panel) and 30 minutes after (bottom panel) the regular daily L37pA peptide gel application. Unlike the data of FIG. 6(b) where the topical peptide was applied to the right hind paw as a control in all mice, in the data of FIG. 7(b), not all mice were provided with topically applied peptide as a control.

As shown in the FIGS. 7(a) and 7(b), the PSNL model showed statistically significant reversal of pain sensation in response to stimuli by topical application of the L37pA peptide as hydrogel or lotion. Formulation without the L37pA peptide was used as a negative control and did not show any pain-reducing activities. The results showed that chronic administration of the L37pA peptide produces sustained pain-relieving effect.

In the PSNL model, repetitive skin application of L37 peptide after PSNL surgery gradually caused sustained reduction of pain. The effect was observed after minimum 3 repetitive (daily) peptide applications. Testing pain 24 hours after the third peptide application showed statistically significant alleviation of neuropathic pain.

Moreover, up to 14 repetitive daily skin applications of vehicle (hydrogel) and L37pA peptide formulation were performed, and in all cases, no adverse effects (general toxicity, behavioral changes, weight loss, skin redness, skin erosions) were noted. Two weeks after the above-described experiments were stopped, the same mice were used in other experiments with intratracheal injection of LPS and HKSA. The mice still responded to the agents normally and those mice without LPS/HKSA treatment were “clean” as the naïve B6 mice as demonstrated by BAL analysis. Chronic topical administration of the L37pA peptide in the therapeutic range of doses does not induce skin irritation or other adverse side effects.

These data show that for the PSNL model of chronic pain, the relieving effect of a single L37pA application 30 minutes before pain assessment was observed at different time points after pain initiation. Additionally, a cumulative pain-relieving effect of repetitive (daily) L37pA treatment was also observed. Therefore, mice receiving daily L37pA skin application retain the pain alleviation effect for next few days, even if L37pA treatment was not performed prior to pain measurements. See “pre-peptide treatment.”

### Example 6: Studies of the Role of the Scavenger Receptor Class B (SRB) in Pathogenesis of Neuroinflammation-Driven Neuropathic Pain

In order to confirm whether to target scavenger receptor class B (SRB) with the SAHPs as a novel therapeutic strategy for neuro-inflammatory pain management, the applicants studied the role of SRBs in pathogenesis of neuroinflammation-driven neuropathic pain.

Besides direct stimulation, sources of neurologically recognized danger signals include both exogenous microorganism-derived, pathogen-associated molecular patterns (PAMPs), and internal, mediated by damage-associated molecular patterns (DAMPs), released by damaged, dying, or necrotic cells in a variety of sterile inflammatory conditions. The critical role of PAMPs and DAMPs in chronic pain is now more recognized since chronic inflammation and elicited inflammatory mediators (IFMs) resulting from PAMP/DAMP-induced activation of pattern recognition receptors (PRRs) at glial cells are known to affect nociceptors. Indeed, IFMs have been demonstrated to activate and sensitize nociceptors directly through IFM receptors located at PNS neurons. There is also a direct pathway in which danger signals (PAMPs/DAMPs) can activate PRRs expressed by nociceptors directly eliciting pain. The most studied group of PRRs is a family of toll-like receptors (TLRs) which were reported in both immune and nociceptor cells of the peripheral nerve system (PNS). It has been demonstrated that TLR activation is mediated by both PAMPs and DAMPs through glial cell and nociceptor neurons by direct and indirect mechanisms. Importantly, PAMPs and DAMPs are also recognized by a variety of host-intrinsic PRRs beyond the TLRs, many of which have been only recently reported as an important part of the danger-sensing process. In contrast to TLRs, other PRRs have not received much attention, especially the class B scavenger receptor (SRB) family proteins, SR-BI, SR-BII, CD36 and LIMP-2, primarily known as lipoprotein receptors. These SRB lipoprotein receptors play important roles in innate immune response, recognizing various pathogens, microorganisms, their PAMPs andacute phase reactants such as SAA, and mediating downstream pro-inflammatory signaling via MAPKs activation, in vitro and in vivo; and may represent a major unrecognized contributor topain.

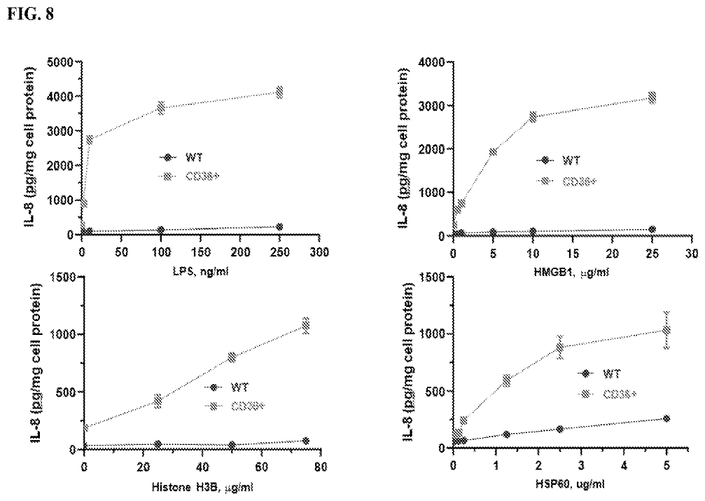

To study this, the applicant conducted cell culture experiments using wildtype (WT) and CD36+ expressing (CD36+) HEK293 cells, treated with increasing amounts of CD36 ligands. Specifically, the ligands tested were the PAMP lipopolysaccharide (LPS) and the DAMPs high mobility group box 1 protein (HMGB1), Histone H3B, and heat shock protein 60 (HSP60). As shown in FIG. 8, in experiments using each of these four ligands, the wildtype HEK293 cells produced a minimal amount of interleukin 8 (IL-8), which is an inflammatory mediator. On the other hand, in experiments using each of these four ligands, the CD36-expressing HEK293 cells produced a significant amount of IL-8, in a dose-dependent manner. This demonstrates that CD36, as a class B scavenger receptor, functions as a PAMP/DAMP sensor, thereby inducing expression of inflammatory mediators such as IL-8. Similarly to HSP60, it is presumed that HSP70 and HSP90 also cause inflammation mediated by the CD36 receptor, and which may be alleviated by the disclosed peptides.

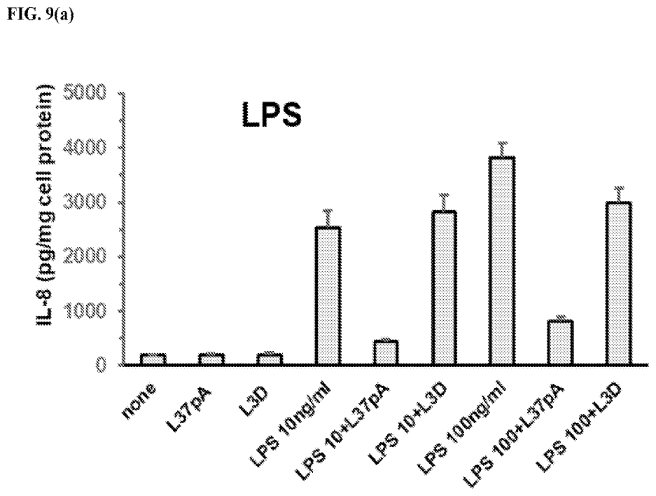

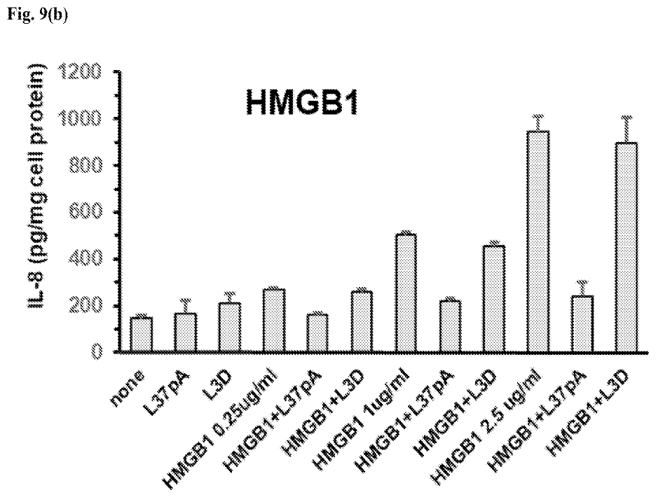

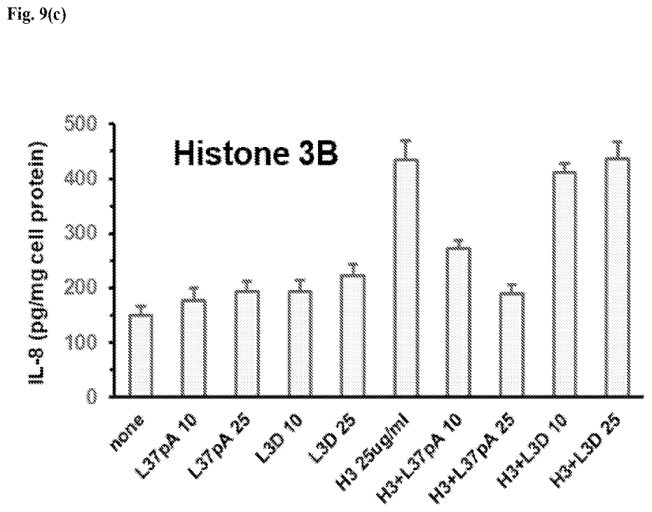

Additionally, the applicant studied the effects of the above-referenced SAHP L37pA as an inhibitor of CD36-dependent, PAMP/DAMP-induced pro-inflammatory response. See FIGS. 9(a)-9(c) To study this, the applicant conducted cell culture experiments using CD36+ HEK293 cells, treated with increasing amounts of CD36 ligands. In FIG. 9(a), the PAMP lipopolysaccharide (LPS) was administered in amounts of 10 ng/ml and 100 ng/ml alone, in combination with 10 μg/ml SAHP L37pA, or in combination with 10 μg/ml SAHP inactive L3D. As controls, no treatment, L37pA alone, and L3D alone were administered. As is clearly shown, L37pA greatly inhibited the production of IL-8 both in the case of 10 ng/ml LPS and 100 ng/ml LPS, relative to administrations of LPS alone. Meanwhile, L3D was ineffective or only slightly effective to inhibit the production of IL-8 relative to LPS alone.

In FIG. 9(b), the DAMP high mobility group box 1 protein (HMGB1) was administered in amounts of 0.25 g/ml, 1 g/ml and 2.5 g/ml alone in combination with 10 μg/ml SAHP L37pA, or in combination with 10 μg/ml SAHP inactive L3D. As controls, no treatment, L37pA alone, and L3D alone were administered. As is clearly shown, L37pA greatly inhibited the production of IL-8 both in the case of 0.25 μg/ml, 1 μg/ml and 2.5 μg/ml HMGB1, relative to administrations of HMGB1 alone. L37pA was especially effective in the case of 1 μg/ml and 2.5 μg/ml HMGB1, relative to administrations of HMGB1 alone. Meanwhile, L3D was ineffective or only slightly effective to inhibit the production of IL-8 relative to HMGB1 alone.

In FIG. 9(c), the DAMP Histone H3B was administered in the amount of 25 μg/ml alone, in combination with 10 μg/ml L37pA, 25 μg/ml L37pA, 10 μg/ml L3D and 25 g/ml L3D. As controls, no treatment, 10 μg/ml L37pA alone, 25 μg/ml L37pA alone, 10 μg/ml L3D alone, and 25 μg/ml L3D alone were administered.

As is clearly shown, L37pA greatly inhibited the production of IL-8 in a dose-dependent manner, with 25 μg/ml L37pA being more effective in inhibiting IL-8 production than 10 μg/ml L37pA, relative to administration of Histone H3B alone. Meanwhile, both at doses of 10 μg/ml and 25 μg/ml L3D was ineffective or only slightly effective to inhibit the production of IL-8 relative to Histone H3B alone.

From this data of FIGS. 9(a) to 9(c), it is concluded that the above-referenced SAHP L37pA can effectively act as an inhibitor of CD36-dependent, PAMP/DAMP-induced pro-inflammatory response. In particular, the SAHPs, as SRB antagonists, efficiently blocked DAMP-mediated IL-8 secretion in HEK236 cells over-expressing CD36.

While the invention has been described with reference to certain particular embodiments thereof, those skilled in the art will appreciate that various modifications may be made without departing from the spirit and scope of the invention. The scope of the appended claims is not to be limited to the specific embodiments described.

1. A method of treating pain in a subject in need thereof, the method comprising administering to the subject a peptide capable of acting as a mimetic of apoA-I protein as defined by SEQ ID NO: 31,
wherein the peptide comprises two sequences selected from the group consisting of SEQ ID NOS: 1, 6, 27, and a variant or derivative thereof, the two sequences being coupled to each other via a proline or an alanine.

2. The method according to claim 1, wherein the peptide comprises a sequence selected from the group consisting of SEQ ID NOS: 2-5, 7-26, 28-30, and a variant or derivative thereof.

3. The method according to claim 1, wherein the peptide comprises SEQ ID NO: 2, 3, 7, 8, 9, or 30.

4. The method according to claim 1, wherein the peptide comprises SEQ ID NO: 2 or 30.

5. The method according to claim 1, wherein the peptide is non-opioid.

6. The method according to claim 1, wherein the pain is acute or chronic pain.

7. The method according to claim 1, wherein the pain is at least one selected from the group consisting of sport-related joint pain, rheumatoid arthritis, burn-related pain, neuropathy, orthopedic pain, phantom pain in amputees and foot neuro-degenerative syndrome.

8. The method according to claim 1, wherein the peptide is administered topically, intravenously, or intramuscularly.

9. The method according to claim 8, wherein the peptide is administered topically as a lotion or a hydrogel.

10. The method according to claim 9, wherein an amount of the peptide in the lotion or the hydrogel is 100 μg/μl to 1000 g/μl.

11. The method according to claim 8, wherein the peptide is administered intramuscularly as an injection, an amount of the peptide in the injection being 0.001 to 100 mg/ml.

12. The method according to claim 1, wherein the peptide is administered once daily.

13. The method according to claim 12, wherein the peptide is administered intramuscularly once daily at a daily dosage of from 0.5 mg/kg to 100 mg/kg body weight.

14. The method according to claim 1, wherein the peptide is comprised in a pharmaceutical composition, the pharmaceutical composition further comprising a pharmaceutically acceptable carrier.

15. The method according to claim 14, wherein the pharmaceutical composition further comprises at least one of a non-steroidal anti-inflammatory drug and an opioid analgesic.

16. A method of treating neuroinflammation in a subject in need thereof, the method comprising administering to the subject a peptide capable of acting as a mimetic of apoA-I protein as defined by SEQ ID NO: 31,
wherein the peptide comprises two sequences selected from the group consisting of SEQ ID NOS: 1, 6, 27, and a variant or derivative thereof, the two sequences being coupled to each other via a proline or an alanine.

17. The method according to claim 16, wherein the peptide consists of a sequence selected from the group consisting of SEQ ID NOS: 2-5, 7-26, 28-30, and a variant or derivative thereof.

18. The method according to claim 16, wherein the peptide comprises SEQ ID NO: 2, 3, 7, 8, 9, or 30.

19. The method according to claim 16, wherein the peptide comprises SEQ ID NO: 2 or 30.

20. The method according to claim 16, wherein the peptide is administered topically, intravenously, or intramuscularly.

21. A method of treating a burn injury in a subject in need thereof, the method comprising administering to the subject a peptide capable of acting as a mimetic of apoA-I protein as defined by SEQ ID NO: 31,
wherein the peptide comprises two sequences selected from the group consisting of SEQ ID NOS: 1, 6, 27, and a variant or derivative thereof, the two sequences being coupled to each other via a proline or an alanine.

22. The method according to claim 21, wherein the peptide consists of a sequence selected from the group consisting of SEQ ID NOS: 2-5, 7-26, 28-30, and a variant or derivative thereof.

23. The method according to claim 21, wherein the peptide comprises SEQ ID NO: 2, 3, 7, 8, 9, or 30.

24. The method according to claim 21, wherein the peptide comprises SEQ ID NO: 2 or 30.

25. The method according to claim 21, wherein the peptide is administered topically, intravenously, or intramuscularly.


SEQUENCE LISTING (31 sequences)

SEQ ID NO: 1 - protein, 18 aa, synthetic construct
DWLKAFYDKVAEKLKEAF

SEQ ID NO: 2 - protein, 37 aa, synthetic construct
DWLKAFYDKVAEKLKEAFPDWLKAFYDKVAEKLKEAF

SEQ ID NO: 3 - protein, 37 aa, synthetic construct
DWLKAFYDKVAEKLKEAFPDWAKAAYDKAAEKAKEAA

SEQ ID NO: 4 - protein, 37 aa, synthetic construct
DHLKAFYDKVACKLKEAFPNWAKAAYDKAAEKAKEAA

SEQ ID NO: 5 - protein, 37 aa, synthetic construct
DWLKAFYDKVAEKLKEAFPDHAKAAYDKAACKAKEAA

SEQ ID NO: 6 - protein, 18 aa, synthetic construct
EKLKELLEKLLEKLKELL

SEQ ID NO: 7 - protein, 37 aa, synthetic construct
EKLKELLEKLLEKLKELLPEKLKELLEKLLEKLKELL

SEQ ID NO: 8 - protein, 37 aa, synthetic construct
EKLLELLKKLLELLKKLLPEKLLELLKKLLELLKKLL

SEQ ID NO: 9 - protein, 37 aa, synthetic construct
EKLKELLEKLLELLKKLLPEKLKELLEKLLELLKKLL

SEQ ID NO: 10 - protein, 37 aa, synthetic construct
EELKEKLEELKEKLEEKLPEELKEKLEELKEKLEEKL

SEQ ID NO: 11 - protein, 37 aa, synthetic construct
EELKAKLEELKAKLEEKLPEELKAKLEELKAKLEEKL

SEQ ID NO: 12 - protein, 37 aa, synthetic construct
EKLKELLEKLKAKLEELLPEKLKELLEKLKAKLEELL

SEQ ID NO: 13 - protein, 37 aa, synthetic construct
EKLKAKLEELKAKLEELLPEKLKAKLEELKAKLEELL

SEQ ID NO: 14 - protein, 37 aa, synthetic construct
EKLKALLEKLLAKLKELLPEKLKALLEKLLAKLKELL

SEQ ID NO: 15 - protein, 37 aa, synthetic construct
EKLKELLEKLLAKLKELLPEKLKELLEKLLAKLKELL

SEQ ID NO: 16 - protein, 37 aa, synthetic construct
EWLKELLEKLLEKLKELLPEWLKELLEKLLEKLKELL

SEQ ID NO: 17 - protein, 37 aa, synthetic construct
EKFKELLEKFLEKFKELLPEKFKELLEKFLEKFKELL

SEQ ID NO: 18 - protein, 37 aa, synthetic construct
EKFKELLEKLLEKLKELLPEKFKELLEKLLEKLKELL

SEQ ID NO: 19 - protein, 37 aa, synthetic construct
EELKELLKELLKKLEKLLPEELKELLKELLKKLEKLL

SEQ ID NO: 20 - protein, 37 aa, synthetic construct
EELKKLLEELLKKLKELLPEELKKLLEELLKKLKELL

SEQ ID NO: 21 - protein, 37 aa, synthetic construct
EKLKELLEKLLEKLKELLAEKLKELLEKLLEKLKELL

SEQ ID NO: 22 - protein, 38 aa, synthetic construct
EKLKELLEKLLEKLKELLAAEKLKELLEKLLEKLKELL

SEQ ID NO: 23 - protein, 37 aa, synthetic construct
DWLKAFYDKVACKLKEAFPDWAKAAYNKAAEKAKEAA

SEQ ID NO: 24 - protein, 37 aa, synthetic construct
DHLKAFYDKVAEKLKEAFPDWAKAAYDKAAEKAKEAA

SEQ ID NO: 25 - protein, 37 aa, synthetic construct
EKLKAKLEELKAKLEELLPEKAKAALEEAKAKAEELA

SEQ ID NO: 26 - protein, 37 aa, synthetic construct
EKLKAKLEELKAKLEELLPEHAKAALEEAKCKAEELA

SEQ ID NO: 27 - protein, 18 aa, synthetic construct
ERLLELLRRLLELLRRLL

SEQ ID NO: 28 - protein, 37 aa, synthetic construct
ERLLELLRRLLELLRRLLPERLLELLRRLLELLRRLL

SEQ ID NO: 29 - protein, 37 aa, synthetic construct
ERLLELLRRLLELLRRLLPDWLKAFYDKVAEKLKEAF

SEQ ID NO: 30 - protein, 37 aa, synthetic construct
EKLLELLKKLLELLKKLLPDWLKAFYDKVAEKLKEAF

SEQ ID NO: 31 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ